SEQUENCE LISTING (13 sequences)

SEQ ID NO: 1 - protein, 728 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 2 - protein, 702 aa, Homo sapiens
MAGFWVGTAPLVAAGRRGRWPPQQLMLSAALRTLKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 3 - protein, 681 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVVAEGQEICVIEAMKMQNSMTAGKTGTVKSVHCQAGDTVGEGDLLVELE

SEQ ID NO: 4 - protein, 539 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 5 - protein, 559 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQQIIGWAQWLPLVISALWEAEDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 6 - protein, 724 aa, Mus musculus
MAGQWVRTVALLAARRHWRRSSQQQLLGTLKHAPVYSYQCLVVSRSLSSVEYEPKEKTFDKILIANRGEIACRVIKTCKKMGIKTVAIHSDVDASSVHVKMADEAVCVGPAPTSKSYLNMDAIMEAIKKTRAQAVHPGYGFLSENKEFAKRLAAEDVTFIGPDTHAIQAMGDKIESKLLAKRAKVNTIPGFDGVVKDADEAVRIAREIGYPVMIKASAGGGGKGMRIAWDDEETRDGFRFSSQEAASSFGDDRLLIEKFIDNPRHIEIQVLGDKHGNALWLNERECSIQRRNQKVVEEAPSIFLDPETRQAMGEQAVALAKAVKYSSAGTVEFLVDSQKNFYFLEMNTRLQVEHPVTECITGLDLVQEMILVAKGYPLRHKQEDIPISGWAVECRVYAEDPYKSFGLPSIGRLSQYQEPIHLPGVRVDSGIQPGSDISIYYDPMISKLVTYGSDRAEALKRMEDALDNYVIRGVTHNIPLLREVIINTRFVKGDISTKFLSDVYPDGFKGHTLTLSERNQLLAIASSVFVASQLRAQRFQEHSRVPVIRPDVAKWELSVKLHDEDHTVVASNNGPAFTVEVDGSKLNVTSTWNLASPLLSVNVDGTQRTVQCLSREAGGNMSIQFLGTVYKVHILTKLAAELNKFMLEKVPKDTSSTLCSPMPGVVVAVSVKPGDMVAEGQEICVIEAMKMQNSMTAGKMGKVKLVHCKAGDTVGEGDLLVELE

SEQ ID NO: 7 - protein, 541 aa, Mus musculus
MAAAIRIRAVAAGARLSVLNCGLGITTRGLCSQPVSVKERIDNKRHAALLGGGQRRIDAQHKRGKLTARERISLLLDPGSFMESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPEVVKSVTNEDVTQEQLGGAKTHTTVSGVAHRAFDNDVDALCNLREFFNFLPLSSQDPAPIRECHDPSDRLVPELDTVVPLESSKAYNMLDIIHAVIDEREFFEIMPSYAKNIVVGFARMNGRTVGIVGNQPNVASGCLDINSSVKGARFVRFCDAFNIPLITFVDVPGFLPGTAQEYGGIIRHGAKLLYAFAEATVPKITVITRKAYGGAYDVMSSKHLLGDTNYAWPTAEIAVMGAKGAVEIIFKGHQDVEAAQAEYVEKFANPFPAAVRGFVDDIIQPSSTRARICCDLEVLASKKVHRPWRKHANIPL

SEQ ID NO: 8 - protein, 504 aa, Mus musculus
MAAAIRIRAVAAGARLSVLNCGLGITTRGLCSQPVSVKERIDNKRHAALLGGGQRRIDAQHKRGKLTARERISLLLDPGSFMESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLDTSYLFITGPEVVKSVTNEDVTQEQLGGAKTHTTVSGVAHRAFDNDVDALCNLREFFNFLPLSSQDPAPIRECHDPSDRLVPELDTVVPLESSKAYNMLDIIHAVIDEREFFEIMPSYAKNIVVGFARMNGRTVGIVGNQPNVASGCLDINSSVKGARFVRFCDAFNIPLITFVDVPGFLPGTAQEYGGIIRHGAKLLYAFAEATVPKITVITRKAYGGAYDVMSSKHLLGDTNYAWPTAEIAVMGAKGAVEIIFKGHQDVEAAQAEYVEKFANPFPAAVRGFVDDIIQPSSTRARICCDLEVLASKKVHRPWRKHANIPL

SEQ ID NO: 9 - DNA, 20 bp, Artificial Sequence
  source: /note=“Description of Artificial Sequence Synthetic primer”
tgggaaaatgggcaaggtga

SEQ ID NO: 10 - DNA, 20 bp, Artificial Sequence
  source: /note=“Description of Artificial Sequence Synthetic primer”
accgaggctccagcctatta

SEQ ID NO: 11 - DNA, 12 bp, Artificial Sequence
  source: /note=“Description of Artificial Sequence Synthetic oligonucleotide”
aagatctgcaaa

SEQ ID NO: 12 - DNA, 12 bp, Artificial Sequence
  source: /note=“Description of Artificial Sequence Synthetic oligonucleotide”
tagagcacagga

SEQ ID NO: 13 - DNA, 14 bp, Artificial Sequence
  source: /note=“Description of Artificial Sequence Synthetic oligonucleotide”
tattgccaataacc

(19) United States
(12) Patent Application Publication
(10) Pub. No.: US 2020/0165593 A1
(43) Pub. Date: May 28, 2020

(54) Modified mRNA Encoding a Propionyl-CoA Carboxylase and Uses Thereof

(71) Applicant: ModernaTX, Inc., Cambridge, MA (US)

(72) Inventors: Susan Sobolov-Jaynes, Essex, CT (US); Romesh R. Subramanian, Framingham, MA (US); Judith L. Campagnari, Westerly, RI (US); Haren Vasavada, Hamden, CT (US); Zhiliang Cheng, Cambridge, MA (US)

(21) Appl. No.: 16/631,607

(22) PCT Filed: Jul. 20, 2018
(86) PCT No.: PCT/US2018/043089

§ 371 Date: Jan. 16, 2020

Related U.S. Application Data
(60) Provisional application No. 62/535,289, filed on Jul. 21, 2017.

(51) Int. Cl. C12N 9/00; C12N 9/88; A61K 31/7115

(52) U.S. Cl. CPC C12N 9/93; C12N 9/88; C12Y 604/01003; C12Y 401/01041; A61K 31/7115

(57) ABSTRACT

Disclosed are methods and compositions for treating propionic academia based on mRNA therapy.

### CROSS-REFERENCE TO RELATED APPLICATIONS

This application claims priority to U.S. Provisional Appl. No. 62/535,289, filed Jul. 21, 2017, the contents of which is incorporated by reference herein in its entirety.

### BACKGROUND

Propionic acidemia (PA) is an autosomal recessive disorder caused by mutations in one or both of the genes encoding propionyl-CoA carboxylase PCCA and PCCB). Proprionyl-CoA (PCC) is a mitochondrial protein complex encoded by nuclear genes. Mutations in the PCC enzyme disrupt the function of the enzyme and prevent normal breakdown of proteins, fat and cholesterol in the body resulting in the accumulation of propionic acid. Biochemically, patients with PA present with elevated levels of PCC, propionic acid, methylcitrate, beta-hydroxy-propionate, propionylglycine, tiglic acid and ketones.

PCC is an enzyme that catalyzes the conversion of propionyl-CoA to methylmalonyl-CoA. PCC comprises of an alpha and beta subunit. The alpha subunit is encoded by the PCCA gene and the beta subunit is encoded by the PCCB gene. Mutations in the PCCA or PCCB gene can result in loss of function or activity of PCCA or PCCB, leading to PA.

The range of PA (also referred to as: PCC deficiency, ketotic glycinemia, hyperglycinemia with ketoacidosis and leukopenia or ketotic hyperglycinemia), ranges from neonatal-onset to late-onset disease. Neonatal-onset PA, the most common form, is characterized by poor feeding, vomiting, and somnolence in the first days of life in a previously healthy infant, followed by lethargy, seizures, coma and death. It is frequently accompanied by metabolic acidosis with anion gap, ketonuria, hypoglycemia, hyperammonemia and cytopenias. Late-onset PA includes developmental regression, chronic vomiting, protein intolerance, failure to thrive, hypotonia, occasionally basal ganglia infarction (resulting in dystonia and choreoathetosis) and cardiomyopathy.

Currently, there is no cure for PA, and only palliative therapies are used for the treatment of PA symptoms (through diet, hemofiltration/hemodialysis, antibiotics and/or liver transplantation). There remains a need to develop compositions and methods for effectively treating PA.

### SUMMARY

Specific embodiments of the invention will become evident from the following more detailed description of certain embodiments and the claims.

In one embodiment, the disclosure is directed to a method of treating propionic acidemia in a patient in need thereof comprising administering to the patient a therapeutically effective amount of a composition comprising a modified mRNA molecule encoding a propionyl CoA carboxylase polypeptide. In a particular embodiment, the modified mRNA molecule encoding a polypeptide comprises at least one of a propionyl CoA carboxylase alpha chain protein or a propionyl CoA carboxylase beta chain protein. In a particular embodiment, the modified mRNA molecule comprises at least one modified nucleoside. In a particular embodiment, the at least one modified nucleoside is selected from the group consisting of: pseudouridine, 1′ methyl-pseudouridine, 5′ methylcytidine, 5′ methyluridine, 2′ O methyluridine, 2′ thiouridine, 5′ methoxyuridine and N6 methyladenosine. In a particular embodiment, the modified mRNA molecule comprises a poly(A) tail, a Kozak sequence, a 3′ untranslated region, a 5′ untranslated region or any combination thereof. In a particular embodiment, the modified mRNA molecule encodes a PCCA subunit comprising a sequence selected from the group consisting of SEQ ID NOS:1-3. In a particular embodiment, the modified mRNA molecule encodes a PCCB subunit comprising a sequence of SEQ ID NO:4 or SEQ ID NO:5. In a particular embodiment, the modified mRNA is encapsulated in a lipid nanoparticle.

In one embodiment, the disclosure is directed to a pharmaceutical composition comprising a therapeutically effective amount of a modified mRNA molecule wherein the modified mRNA molecule encodes one or both of a propionyl CoA carboxylase subunit. In a particular embodiment, the proprionyl CoA carboxylase is an alpha chain protein comprising the amino acid sequence selected from the group consisting of SEQ ID NOS:1 3, and a pharmaceutically acceptable carrier, diluent or excipient. In a particular embodiment, the proprionyl CoA carboxylase is an beta chain protein comprising the amino acid sequence of SEQ ID NO:4 or SEQ ID NO:5, and a pharmaceutically acceptable carrier, diluent or excipient.

### BRIEF DESCRIPTION OF THE DRAWINGS

FIGS. 1A-1B demonstrate PCC mRNA and protein levels in immortalized cellular models. FIG. 1A is a graph showing mRNA expression (calculated as the relative expression to GAPDH levels) of PCCA (the upper panel) and PCCB (the lower panel) in various cell lines. FIG. 1B depicts a western blot showing PCCA, PCCB and GAPDH (control) protein expression in various cell lysates (including hepatoma cell lines HepG2, Hep3B, and SNU-475, common control cell lines HeLa, NIH-3T3 and HEK293, and putative homozygous deletion cell lines Calu3, H2291, H522, and HPAFII).

FIG. 2A depicts the PCCA and PCCB protein expression levels (by western blot) in PCCA-deficient patient lymphoblastoid cell lines (LCLs). FIG. 2A: lines 1-3 represent healthy LCL #1-3, respectively; lines 4-5 represent cell lines from parents of PA patients #1-2, respectively; lines 6-10 represent cell lines from PA patients #1-5, respectively. FIG. 2B is a detailed table describing genotypes of cell lines shown in FIG. 2A.

FIG. 3A depicts the PCCA protein expression levels (by western blot) in PCCA or PCCB-deficient patient fibroblasts (9 cell lines). FIG. 3B is a detailed table describing genotypes of cell lines shown in FIG. 3A.

FIG. 4A depicts the endogenous PCCA and PCCB protein expression in normal (+/+; wild-type), clinically unaffected parent (+/mt; heterozygous for PCCA mutation) and PA patient (mt/mt; homozygous for PCCA mutation) fibroblasts. FIG. 4B depicts the activity (represented by ¹⁴C-bicarbonate fixation activity) of endogenous PCC complex in these fibroblasts.

FIGS. 5A-5B depict the PCCA and PCCB protein levels in multiple immortalized cells after transfection of PCCA DNA. FIG. 5A and FIG. 5B represent the western blot analyses of two experiments in different cells. “Vec” represents control cells transfected with empty plasmid vector.

FIGS. 6A-6B depict PCCA/B protein levels in patient fibroblasts (FIG. 6A) and lymphoblastoid cells (LCLs) (FIG. 6B) after transfection of PCCA DNA. “Ctrl” represents control cells transfected with empty plasmid vector.

FIGS. 7A-7B depict modified PCCA mRNA (modRNA) restored and stabilized PCCB levels in human PA patient fibroblasts. FIGS. 7A and 7B depict a western blot showing PCCA, PCCB and GAPDH (control) expression in human PA patient fibroblasts transfected with LX-hPCCA01 modRNA or luciferase modRNA. Lysates were harvested 24 hours after transfection.

FIG. 8 depicts a western blot demonstrating PCCA, PCCB and GAPDH (control) expression in human PA patient fibroblasts following transfection of a modified mRNA molecule at concentrations of 250 ng-5000 ng.

FIGS. 9A and 9B demonstrate that modified human PCCA (hPCCA) mRNA and its FLAG-tagged variants reconstituted PCC activity in human PCCA-deficient patient fibroblasts. FIG. 9A depicts a western blot showing PCCA, FLAG, PCCB and GAPDH (control) expression in human PCCA-deficient patient fibroblasts transfected with modified hPCCA mRNA or its FLAG-tagged variant. FIG. 9B is a graph illustrating PCC enzyme activity on tagged variants.

FIGS. 10A-10C demonstrate localization of transfected modified hPCCA mRNA to the mitochondria in mouse fibroblasts. FIG. 10A shows the co-localization of 21988-1-AP (1:500 dilution), which identifies the expressed hPCCA mRNA, and anti-rabbit Alexa 488 (1:1000), which identifies the mitochondria in human cells transfected with hPCCA. FIG. 10B shows the co-localization of 21988-1-AP (1:500 dilution), which identifies the expressed hPCCA mRNA and anti-rabbit Alexa 488 (1:1000), which identifies the mitochondria in mouse cells transfected with hPCCA. FIG. 10C shows the co-localization of 21988-1-AP (1:500 dilution) and anti-rabbit Alexa 488 (1:1000) in untransfected control cells.

FIGS. 11A-11C demonstrate sustained PCCA and PCCB expression five days post-transfection of modified PCCA mRNA. FIG. 11A depicts a western blot showing PCCA, PCCB and vinculin (control) expression in cells at six hours to five days post transfection with modified PCCA mRNA. FIGS. 11B and 11C are graphs showing the total RNA and protein levels of PCCA (FIG. 11B) and PCCB (FIG. 11C) at six hours to five days post-transfection with modified PCCA mRNA.

FIG. 12 depicts a western blot showing PCCA overexpression from modRNA in patient fibroblasts. Cells were transfected with empty vector control (ctrl), untagged PCCA (no tag), two different versions of N-terminal FLAG-tagged PCCA (N-V1 and N-V2), and one C-terminal FLAG-tagged PCCA (C-term). Antibodies recognizing PCCA (which detects both human and mouse PCCA), FLAG tag, and GAPDH were used. The blot with anti-FLAG antibody was analyzed with short-time (short) or long-time (long exp) exposure for the blot reaction.

FIGS. 13A and 13B depict western blots showing PCCA, PCCB and GAPDH (control) expression in wild-type mouse hepatocytes transfected with modified hPCCA constructs (FIG. 13A) or modified mouse PCCA constructs (FIG. 13B).

FIGS. 14A-14C depict western blots showing PCCA, PCCB and GAPDH (control) expression in PA patient fibroblasts (GM371) transfected with modified hPCCA (FIG. 14A), hPCCA with a N-terminal FLAG tag variant 2 (FIG. 14B) or hPCCA with a C-terminal FLAG (FIG. 14C) at 0-14 days post-transfection.

FIGS. 15A and 15B depict western blots showing PCCA and vinculin (control) expression in crude liver lysates from five wild-type mice administered non-translating Factor IX modified hPCCA with an N-terminal FLAG variant 2 and hPCCA with a C-terminal FLAG.

FIG. 16 depicts a western blot showing PCCA, GAPDH and COX IV expression in crude liver lysates and mitochondrial fractions from wild-type mice administered non-translating Factor IX or modified hPCCA with a C-terminal FLAG.

FIGS. 17A-17D demonstrate that modified PCCA protein was detected in liver mitochondria up to seven days post injection of mouse PCCA. FIG. 17A depicts western blots showing PCCA, PCCB, and HSP60 expression in liver mitochondrial fractions from wild-type mice administered non-translating Factor IX (ntFIX) and mouse modified hPCCA mRNA at 24-168 hours post-injection. FIGS. 17B-17D are graphs showing quantification of PCCA and HSP60 0-8 days post-injection with 2.5 mg/kg ntFIX control, 0.5 mg/pk mPCCA modRNAs, or 2.5 mg/pk mPCCA modRNAs.

FIGS. 18A and 18B demonstrate mouse-modified PCCA decay kinetics in wild-type mouse liver. FIG. 18A depicts a graph demonstrating levels of mouse-modified PCCA mRNA (injected in a 0.5 mg/kg or 2.5 mg/kg dosage) in the liver of wild-type mice 0-200 hours post-injection. FIG. 18B depicts a graph demonstrating the total levels of PCCA mRNA in the liver of wild-type mice 0-200 hours post-injection of ntFIX or modified PCCA mRNA.

FIGS. 19A-19C demonstrate reduced PCC complex expression in A138T mouse hypomorphic model. FIG. 19A depicts a western blot showing PCCA, PCCB and vinculin expression in the A138T hypomorphic mouse model. FIGS. 19B and 19C are graphs illustrating normalized PCCA and PCCB protein levels in the A138T hypomorphic mouse model.

FIGS. 20A-20C depicts PCC expression in A138T mice treated with human or mouse PCCA-LNP constructs at 48 hours post injection. FIG. 20A depicts a western blot showing PCCA, PCCB, and GAPDH (control) expression in mouse livers of each cohort. WT FVB mice were used as control. FIG. 20B is a graph summarizing PCCA and PCCB protein levels in A138T mice (with a PCCA^{-/-}; A138T+^{+/+} genotype) from the experiments in FIG. 20A. FIG. 20C depicts the dosage-related overexpression of exogenous hPCCA-FLAG proteins in treated A138T mice.

FIG. 21 is a graph illustrating the overexpression of exogenous human or mouse PCCA proteins (untagged or C-terminal FLAG-tagged) in A138T mice. 40 μg of protein was loaded to the assay reaction system for homogenates. The result was normalized to protein concentration. Each sample was assayed in duplicate.

FIG. 22 depicts the blood 2-methylcitric acid (2-MC) levels with or without i.v. injection of hPCCA or mPCCA modRNA constructs. FIG. 22A is a graph illustrating the blood 2-MC concentration pre- or 48 hours post injection for each animal in different cohorts. FIG. 22B is a graph illustrating the average % change in 2-MC concentrations. FIG. 22C is a graph illustrating the % change in 2-MC concentrations for each animal of different cohorts.

FIGS. 23A-23C depict the blood propionylcarnitine (C3) levels with or without i.v. injection of hPCCA or mPCCA modRNA constructs. FIG. 23A is a graph illustrating the blood C3 concentration pre- or 48 hours post injection for each animal in different cohorts. FIG. 23B is a graph illustrating the average % change in C3 concentrations. FIG. 23C is a graph illustrating the % change in C3 concentrations for each animal of different cohorts. * p<0.05

FIGS. 24A-24C depict the ratio of propionylcarnitine (C3)/acetylcarnitine (C2) blood levels with or without i.v. injection of hPCCA or mPCCA modRNA constructs. FIG. 24A is a graph illustrating the blood C3/C2 ratio pre- or 48 hours post injection for each animal in different cohorts. FIG. 24B is a graph illustrating the average change in C3/C2 ratios. FIG. 24C is a graph illustrating the % change in C3/C2 ratios for each animal of different cohorts. * p<0.05

FIGS. 25A-25B depict the plasma 2-methylcitric acid (2-MC) levels with or without i.v. injection of hPCCA or mPCCA modRNA constructs. FIG. 25A is a graph illustrating the plasma 2-MC concentration pre- or 48 hours post injection for each animal in different cohorts. FIG. 25B is a graph illustrating the average % change in 2-MC concentrations.

FIGS. 26A-26B depict the plasma 3-hydroxypropionate (3-HP) levels with or without i.v. injection of hPCCA or mPCCA modRNA constructs. FIG. 26A is a graph illustrating the plasma 3-HP concentration pre- or 48 hours post injection for each animal in different cohorts. FIG. 26B is a graph illustrating the average % change in 3-HP concentrations. * p<0.05

FIGS. 27A-27B depict the plasma C3 levels with or without i.v. injection of hPCCA or mPCCA modRNA constructs. FIG. 27A is a graph illustrating the plasma C3 concentration pre- or 48 hours post injection for each animal in different cohorts. FIG. 27B is a graph illustrating the average % change in C3 concentrations. * p<0.05

FIGS. 28A-28B depict the plasma C3/C2 ratio with or without i.v. injection of hPCCA or mPCCA modRNA constructs. FIG. 28A is a graph illustrating the plasma C3/C2 ratio pre- or 48 hours post injection for each animal in different cohorts. FIG. 28B is a graph illustrating the average % change in C3/C2 ratio. * p<0.05

FIG. 29A depicts the standard curve of detecting C2 (acetylcarnitine) (with different concentrations at room temperature for 9.3 min). At low concentrations, the total area is in direct proportion to C2 concentration (FIG. 29B).

FIG. 30 depicts the detection of C2 at different concentration standards by liquid chromatography-mass spectrometry (LC-MS) (SIM).

FIGS. 31A and 31B depict the detection of C3 at different plasma concentrations by liquid chromatography-mass spectrometry (LC-MS) (SIM).

FIG. 32A depicts a Western blot image showing the overexpression of PCCA and PCCB protein levels in A138T hypomorphic mice treated with modRNA constructs. FIG. 32B quantifies and illustrates the ratio of such overexpression to wild type levels.

FIG. 33 is a graph depicting the effect of PCCA and PCCB expression on PCC activity.

### DETAILED DESCRIPTION

Provided herein are nucleic acid molecules, including modified nucleic acid molecules, and methods of using the same. The nucleic acid molecules, including RNAs such as mRNAs, contain, for example, one or more modifications that improve properties of the molecule. Such improvements include, but are not limited to, increased stability and/or clearance in tissues, improved receptor uptake and/or kinetics, improved cellular access by the compositions, improved engagement with translational machinery, improved mRNA half-life, increased translation efficiency, improved immune evasion, improved protein production capacity, improved secretion efficiency, improved accessibility to circulation, improved protein half-life and/or modulation of a cell's status, improved function and/or improved activity.

The present disclosure provides compositions of nucleic acids capable of regulating protein expression of propionyl-CoA carboxylase (PCC) or a biologically active fragment thereof in a target cell. In addition, methods and processes of preparing and delivering such nucleic acid to a target cell are also provided. Furthermore, kits and devices for the design, preparation, manufacture and formulation of such nucleic acids are also included in the instant disclosure. The compositions provided herein are useful for treating diseases or disorder associated with a deficiency of PCC activity, such as, for example, propionic acidemia (PA). Nucleic acids include, for example, polynucleotides, which further include, for example, ribonucleic acids (RNAs), deoxyribonucleic acids (DNAs), threose nucleic acids (TNAs; Yu, H. et al., Nat. Chem., 4:183-7, 2012), glycol nucleic acids (GNAs, for reviews see Ueda, N. et al., J. Het. Chem., 8:827-9, 1971; Zhang, L. et al., J. Am. Chem. Soc., 127:4174-5, 2005), peptide nucleic acids (PNAs; Nielsen, P. et al., Science, 254:1497-500, 1991), locked nucleic acids (LNAs; Alexei, A. et al., Tetrahedron, 54:3607-30, 1998), and other polynucleotides known in the art.

The nucleic acid molecule can be, for example, a messenger RNA (mRNA). In some embodiments, the mRNA encodes a PCC (e.g., PCCA and PCCB) or a biologically active fragment thereof. In one embodiment, the mRNA is delivered into a target cell to express at least one PCC subunit (e.g., the alpha subunit (PCCA) and/or the beta subunit (PCCB)) or a biologically active fragment thereof in vivo, in situ or ex vivo. In another embodiment, the mRNA is delivered into an animal, e.g., a mammal (such as a human), to express such at least one subunit or a biologically active fragment thereof. The mRNA provided can treat or alleviate a symptom, a disease or a disorder associated with a deficiency of PCC activity, such as, propionic acidemia (PA).

### RNA Structure

Modified mRNA molecules are described herein that provide for a therapeutic tool for use in enzyme replacement therapy (ERT), e.g., for treating PA or a disease or condition associated with PCC deficiency. The terms “modified” or “modification” as used herein refer to an alteration of a nucleic acid residue that can be, for example, incorporated into a polynucleotide, e.g., an mRNA molecule, that can then be used for a therapeutic treatment. Modifications to an mRNA molecule can include, for example, physical or chemical modifications to a base, such as, for example, the depletion of a base or a chemical modification of a base, or sequence modifications to a nucleic acid sequence relative to a reference nucleic acid sequence.

Described herein are compositions for modulating the expression of a PCC (e.g., PCCA and/or PCCB) or a biologically active fragment thereof in vitro or in vivo, e.g., in a target cell. The mRNA molecule can, for example, replace, increase or promote expression of such a PCC or biologically active fragment thereof. In some embodiments, the composition comprises an artificially synthesized or isolated nature RNA molecule with or without a transfer vehicle. An RNA molecule can comprise, for example, a sequential series of sequence elements, wherein, for example, sequence C comprises a nucleic acid sequence encoding a PCC or a biologically active fragment thereof. C may comprise, with or without a bridging linker (such as a peptide linker comprising at least one amino acid residue), one or more 5′ signal sequence(s). A sequence B, upstream of C, can comprise an optional flanking region comprising one or more complete or incomplete 5′ untranslated region (UTR) sequences. A sequence A, upstream of B, can comprise an optional 5′ terminal cap. A sequence D, downstream of C, can comprise an optional flanking region comprising one or more complete or incomplete 3′ UTR sequences. A sequence E, downstream of D, can comprise an optional flanking region comprising a 3′ tailing sequence. Bridging the 5′ terminus of C and the flanking sequence B is an optional first operational region. This first operational region traditionally comprises a start codon. The operational region can also comprise, for example, a translation initiation sequence or signal sequence. Bridging the 3′ end of C and the flanking region D is an optional second operational region. This second operational region can comprise, for example, a stop codon. The operational can also comprise a translation termination sequence or signal sequence. Multiple, serial stop codons can also be used. Sequence E can comprise a 3′ tail sequence, e.g., a poly A tail.

UTRs are transcribed but not translated. The 5′ UTR starts at the transcription start site and continues to the start codon but does not include the start codon; whereas, the 3′ UTR starts immediately following the stop codon and continues until the transcriptional termination signal. Natural 5′ UTRs help translation initiation, and they comprise features such as, for example, Kozak sequences, which facilitate translation initiation by the ribosome for many genes. Kozak sequences have the consensus CCR(A/G)CCAUGG, where R is a purine (adenine or guanine) three bases upstream of the start codon (AUG), which is followed by another G.

3′ UTRs are rich in adenosines and uridines. These AU rich signatures are particularly prevalent in genes with high rates of turnover. Based on their sequence features and functional properties, the AU rich elements (AREs) can be separated into three classes—Class I AREs (such as those in c-Myc and MyoD) contain several dispersed copies of an AUUUA motif within U rich regions; Class II AREs possess two or more overlapping UUAUUUA(U/A)(U/A) nonamers (molecules containing this type of ARE include GM-CSF and TNFα); Class III ARES are less well defined (these U rich regions do not contain an AUUUA motif; c-Jun and myogenin are two examples of this class). Most proteins binding to the AREs destabilize the messenger, whereas members of the ELAV family, most notably HuR, increase the stability of mRNA. Engineering HuR specific binding site(s) into the 3′ UTR of the mRNA leads to HuR binding and thus, stabilization of the mRNA.

Introduction, removal or modification of 3′ UTR AREs can be used to modulate the stability of mRNA. When engineering specific mRNA, one or more copies of an ARE can be introduced to make such mRNA less stable and thereby curtail translation and decrease production of the resultant protein. Likewise, AREs can be identified and removed or mutated to increase the intracellular stability and thus increase translation and production of the resultant protein.

The 5′ cap structure of an mRNA is involved in nuclear export and mRNA stability in the cell. The cap binds to Cap Binding Protein (CBP), which is responsible for in vivo mRNA stability and translation competency through the interaction of CBP with poly-A binding protein to form the mature cyclic mRNA species. The cap further assists the removal of 5′ proximal introns during mRNA splicing. The mRNA molecules described herein can be 5′ end capped to generate a 5′-ppp-5′ triphosphate linkage. The linkage site can be, for example, between a terminal guanosine cap residue and the 5′-terminal transcribed sense nucleotide of the mRNA molecule. This 5′-guanylate cap may then be methylated to generate an N7 methyl guanylate residue. The ribose sugars of the terminal and/or anteterminal transcribed nucleotides of the 5′ end of the mRNA may optionally also be 2′-O-methylated. 5′ decapping through hydrolysis and cleavage of the guanylate cap structure may target a nucleic acid molecule, such as an mRNA molecule, for degradation.

mRNA can be capped post transcriptionally, for example, using enzymes to generate more authentic 5′ cap structures. As used herein, the phrase “more authentic” refers to a feature that closely mirrors or mimics, either structurally or functionally, a naturally occurring feature. That is, a “more authentic” feature is better representative of physiological cellular function and/or structure as compared to synthetic features or analogs. Non limiting examples of more authentic 5′ cap structures are those that, among other things, have enhanced binding of CBPs, increased half-life, reduced susceptibility to 5′ endonucleases and/or reduced 5′ decapping, as compared to synthetic 5′ cap structures. Recombinant Vaccinia virus capping enzyme and recombinant 2′-O-methyltransferase, for example, can create a canonical 5′-5′-triphosphate linkage between the 5′ terminal nucleotide of an mRNA and a guanine cap nucleotide wherein the cap guanine contains an N7 methylation and the 5′ terminal nucleotide of the mRNA contains a 2′-O-methyl. Such a structure is termed the “Cap1” structure. This cap results in a higher translational competency and cellular stability and a reduced activation of cellular pro-inflammatory cytokines, as compared, for example, to other 5′ cap analog structures. Because the mRNA of the instant disclosure may be capped post transcriptionally, and because this process is more efficient, nearly 100% of the mRNA may be capped. This is in contrast to the ˜80% capping rate when a cap analog is linked to an mRNA in the course of an in vitro transcription reaction.

Cap analogs can be used to modify the 5′ end of an mRNA molecule. Cap analogs, synthetic cap analogs, chemical caps, chemical cap analogs, or structural or functional cap analogs, differ from natural 5′ caps in their chemical structure, while still retaining cap function. Cap analogs can be chemically or enzymatically synthesized and/or linked to the mRNA, e.g., modRNA, described herein. The Anti Reverse Cap Analog (ARCA), for example, contains two guanines linked by a 5′-5′-triphosphate group, wherein one guanine contains an N7 methyl group as well as a 3′-O-methyl group. Another exemplary cap is mCAP, which is similar to ARCA but has a 2′-O-methyl group on guanosine. Cap structures include, but are not limited to, 5′ triphosphate cap (5′-ppp), Guanosine triphosphate Cap (5′-Gppp), 5′ N7-methylguanosine-triphosphate Cap (5′-N7-MeGppp, 7mGppp), 5′ adenylated cap (rApp), 7mG(5′)ppp(5′)N, pN2p (cap 0), 7mG(5′)ppp(5′)NlmpNp (cap 1), and 7mG(5)-ppp(5′)NlmpN2mp (cap 2) (Konarska, M. et al., Cell, 38:731-6, 1984; the entire contents of which are incorporated by reference). A 5′ terminal cap can further comprise a guanine analog. Useful guanine analogs include, but are not limited to, inosine, N1-methyl guanosine, 2′-fluoro guanosine, 7-deaza guanosine, 8-oxo guanosine, 2-amino guanosine, LNA guanosine and 2-azido guanosine.

### RNA Sequence

The instant disclosure provides mRNA sequences encoding at least one of Propionyl-CoA carboxylase subunits or a biologically active fragment thereof, which is useful for, among other things, treating a disease or disorder associated with a deficiency of Propionyl-CoA carboxylase activity, such as PA. As used herein, a “biologically active fragment” refers to a portion of a molecule, e.g., a gene, coding sequence, mRNA, polypeptide or protein, which has a desired length or biological function. A biologically active fragment of a protein, for example, can be a fragment of the full-length protein that retains one or more biological activities of the protein. A biologically active fragment of an mRNA, for example, can be a fragment that, when translated, expresses a biologically active protein fragment. A biologically active mRNA fragment, furthermore, can comprise shortened versions of non-coding sequences, e.g., regulatory sequences, UTRs, etc. In general, a fragment of an enzyme or signaling molecule can be, for example, that portion(s) of the molecule that retains its signaling or enzymatic activity. A fragment of a gene or coding sequence, for example, can be that portion of the gene or coding sequence that produces an expression product fragment. As used herein, “gene” is a term used to describe a genetic element that gives rise to expression products (e.g., pre-mRNA, mRNA, polypeptides etc.). A fragment does not necessarily have to be defined functionally, as it can also refer to a portion of a molecule that is not the whole molecule, but has some desired characteristic or length (e.g., restriction fragments, amplification fragments, etc.).

Additional sequence modification, for example to the 3′ UTR, include the insertion of, for example, viral sequences such as the translation enhancer sequence of the barley yellow dwarf virus (BYDV PAV), the Jaagsiekte sheep retrovirus (JSRV) and/or the Enzootic nasal tumor virus (PCT Pub. No. WO2012129648; herein incorporated by reference in its entirety).

Modified mRNA (modRNA) described herein can comprise an internal ribosome entry site (IRES). IRESs play an important role in initiating protein synthesis in absence of the 5′ cap structure. An IRES can act as the sole ribosome binding site, or serve as one of multiple ribosome binding sites of an mRNA. An mRNA containing more than one functional ribosome binding site can encode several peptides or polypeptides that are translated independently by the ribosomes (“multicistronic nucleic acid molecules”). A modRNA can thus encode, for example, multiple portions or fragments of a PCC or a biologically active fragment thereof. Examples of IRES sequences that can be used include IRESs derived from, for example, picornaviruses (e.g., FMDV), pest viruses (CFFV), polio viruses (PV), encephalomyocarditis viruses (ECMV), foot and mouth disease viruses (FMDV), hepatitis C viruses (HCV), classical swine fever viruses (CSFV), murine leukemia virus (MLV), simian immune deficiency viruses (SIV) and cricket paralysis viruses (CrPV).

During RNA processing, a long chain of adenine nucleotides (poly-A tail) can be added to the mRNA molecule. The process, called polyadenylation, adds a poly-A tail that can be between, for example, about 100 and 250 residues long. In some embodiments, unique poly-A tail lengths provide certain advantages to the mRNA of the instant disclosure. Generally, the length of a poly-A tail is greater than 30 nucleotides in length (e.g., at least or greater than about 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA comprises a poly-A tail of a length from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000). In some embodiments, the poly-A tail is designed relative to the length of the overall mRNA. This design may be based on the length of the coding region, the length of a particular feature or region (such as the first or flanking regions), or based on the length of the ultimate product expressed from the mRNA. The poly-A tail can be, for example, 10, 20, 30, 40, 50, 60, 70, 80, 90 or 100% greater in length than the rest of the mRNA sequence. The poly-A tail can also be designed as a fraction of such mRNA.

mRNA can be linked together to the Poly A binding protein (PABP) through the 3′ end using modified nucleotides at the 3′ terminus of the poly-A tail. In one embodiment, mRNA can include a poly-A tail G quartet. The G quartet is a cyclic hydrogen bonded array of four guanine nucleotides that can be formed by G rich sequences in both DNA and RNA. In this embodiment, the G quartet is incorporated at the end of the poly-A tail.

Other RNA sequence modification elements and methods include a combination of nucleotide modifications abrogating mRNA interaction with Toll like receptor 3 (TLR3), TLR7, TLR8 and retinoid inducible gene 1 (RIG 1), resulting in low immunogenicity and higher stability in mice (Kormann, M. et al., Nat. Biotechnol., 29:154-7, 2011; the content of which is incorporated by reference herein in its entirety).

### Propionyl-CoA Carboxylase (PCC)

PCC is a biotin-dependent enzyme capable of catalyzing the carboxylation reaction of propionyl CoA in the mitochondrial matrix. The product of the reaction is (S)-methylmalonyl CoA. Propionyl CoA is the end product of metabolism of odd-chain fatty acids, and a metabolite of most methyl-branched fatty acids. PCC is a 750 kDa dodecamer comprising six alpha (α) subunits (PCCA) and six beta (β) subunits (PCCB). The alpha subunits are arranged as monomers, decorating the central beta-6 hexameric core. Said core is oriented as a short cylinder with a hole along its axis (Kalousek, F. et al., J. Biol. Chem., 255:60-5, 1980). The alpha subunit of PCC contains the biotin carboxylase (BC) and biotin carboxyl carrier protein (BCCP) domains. A domain known as the BT domain is also located on the alpha subunit and is essential for interactions with the beta subunit. The beta subunit contains the carboxyltransferase (CT) activity (Diacovich, L. et al., Biochemistry, 43:14027-36, 2004).

Exemplary mRNA sequences encoding human PCCA are published as NCBI reference nos. NM_000282 (isoform a), NM_001127692 (isoform b), and NM_001178004 (isoform c). Exemplary protein sequences of PCCA are published as NCBI reference nos. NP_000273 (isoform a, SEQ ID NO: 1), NP_001121164 (isoform b, SEQ ID NO: 2), and NP_001171475 (isoform c, SEQ ID NO: 3). For a complete summary of human PCCA genomic sequence and other information, see NCBI database Gene ID: 5095.

(SEQ ID NO: 1)
MAGFWVGTAP LVAAGRRGRW PPQQLMLSAA LRTLKHVLYY
SRQCLMVSRN LGSVGYDPNE KTFDKILVAN RGEIACRVIR
TCKKMGIKTV AIHSDVDASS VHVKMADEAV CVGPAPTSKS
YLNMDAIMEA IKKTRAQAVH PGYGFLSENK EFARCLAAED
VVFIGPDTHA IQAMGDKIES KLLAKKAEVN TIPGFDGVVK
DAEEAVRIAR EIGYPVMIKA SAGGGGKGMR IAWDDEETRD
GFRLSSQEAA SSFGDDRLLI EKFIDNPRHI EIQVLGDKHG
NALWLNEREC SIQRRNQKVV EEAPSIFLDA ETRRAMGEQA
VALARAVKYS SAGTVEFLVD SKKNFYFLEM NTRLQVEHPV
TECITGLDLV QEMIRVAKGY PLRHKQADIR INGWAVECRV
YAEDPYKSFG LPSIGRLSQY QEPLHLPGVR VDSGIQPGSD
ISIYYDPMIS KLITYGSDRT EALKRMADAL DNYVIRGVTH
NIALLREVII NSRFVKGDIS TKFLSDVYPD GFKGHMLTKS
EKNQLLAIAS SLFVAFQLRA QHFQENSRMP VIKPDIANWE
LSVKLHDKVH TVVASNNGSV FSVEVDGSKL NVTSTWNLAS
PLLSVSVDGT QRTVQCLSRE AGGNMSIQFL GTVYKVNILT
RLAAELNKFM LEKVTEDTSS VLRSPMPGVV VAVSVKPGDA
VAEGQEICVI EAMKMQNSMT AGKTGTVKSV HCQAGDTVGE
GDLLVELE
(SEQ ID NO: 2)
MAGFWVGTAP LVAAGRRGRW PPQQLMLSAA LRTLKTFDKI
LVANRGEIAC RVIRTCKKMG IKTVAIHSDV DASSVHVKMA
DEAVCVGPAP TSKSYLNMDA IMEAIKKTRA QAVHPGYGFL
SENKEFARCL AAEDVVFIGP DTHAIQAMGD KIESKLLAKK
AEVNTIPGFD GVVKDAEEAV RIAREIGYPV MIKASAGGGG
KGMRIAWDDE ETRDGFRLSS QEAASSFGDD RLLIEKFIDN
PRHIEIQVLG DKHGNALWLN ERECSIQRRN QKVVEEAPSI
FLDAETRRAM GEQAVALARA VKYSSAGTVE FLVDSKKNFY
FLEMNTRLQV EHPVTECITG LDLVQEMIRV AKGYPLRHKQ
ADIRINGWAV ECRVYAEDPY KSFGLPSIGR LSQYQEPLHL
PGVRVDSGIQ PGSDISIYYD PMISKLITYG SDRTEALKRM
ADALDNYVIR GVTHNIALLR EVIINSRFVK GDISTKFLSD
VYPDGFKGHM LTKSEKNQLL AIASSLFVAF QLRAQHFQEN
SRMPVIKPDI ANWELSVKLH DKVHTVVASN NGSVFSVEVD
GSKLNVTSTW NLASPLLSVS VDGTQRTVQC LSREAGGNMS
IQFLGTVYKV NILTRLAAEL NKFMLEKVTE DTSSVLRSPM
PGVVVAVSVK PGDAVAEGQE ICVIEAMKMQ NSMTAGKTGT
VKSVHCQAGD TVGEGDLLVE LE
(SEQ ID NO: 3)
MAGFWVGTAP LVAAGRRGRW PPQQLMLSAA LRTLKHVLYY
SRQCLMVSRN LGSVGYDPNE KTFDKILVAN RGEIACRVIR
TCKKMGIKTV AIHSDVDASS VHVKMADEAV CVGPAPTSKS
YLNMDAIMEA IKKTRAQAVH PGYGFLSENK EFARCLAAED
VVFIGPDTHA IQAMGDKIES KLLAKKAEVN TIPGFDGVVK
DAEEAVRIAR EIGYPVMIKA SAGGGGKGMR IAWDDEETRD
GFRLSSQEAA SSFGDDRLLI EKFIDNPRHI EIQVLGDKHG
NALWLNEREC SIQRRNQKVV EEAPSIFLDA ETRRAMGEQA
VALARAVKYS SAGTVEFLVD SKKNFYFLEM NTRLQVEHPV
TECITGLDLV QEMIRVAKGY PLRHKQADIR INGWAVECRV
YAEDPYKSFG LPSIGRLSQY QEPLHLPGVR VDSGIQPGSD
ISIYYDPMIS KLITYGSDRT EALKRMADAL DNYVIRGVTH
NIALLREVII NSRFVKGDIS TKFLSDVYPD GFKGHMLTKS
EKNQLLAIAS SLFVAFQLRA QHFQENSRMP VIKPDIANWE
LSVKLHDKVH TVVASNNGSV FSVEVDGSKL NVTSTWNLAS
PLLSVSVDGT QRTVQCLSRE AGGNMSIQFL GTVVAEGQEI
CVIEAMKMQN SMTAGKTGTV KSVHCQAGDT VGEGDLLVEL E

Exemplary mRNA sequences encoding human PCCB are published as NCBI reference nos. NM_000532 (isoform 1) and NM_001178014 (isoform 2). Exemplary protein sequences of human PCCB are published as NCBI reference nos. NP_000523 (isoform 1, SEQ ID NO: 4) and NP_001171485 (isoform 2, SEQ ID NO: 5). For a complete summary of human PCCB genomic sequence and other information, see NCBI database Gene ID: 5096.

(SEQ ID NO: 4)
MAAALRVAAV GARLSVLASG LRAAVRSLCS QATSVNERIE
NKRRTALLGG GQRRIDAQHK RGKLTARERI SLLLDPGSFV
ESDMFVEHRC ADFGMAADKN KFPGDSVVTG RGRINGRLVY
VFSQDFTVFG GSLSGAHAQK ICKIMDQAIT VGAPVIGLND
SGGARIQEGV ESLAGYADIF LRNVTASGVI PQISLIMGPC
AGGAVYSPAL TDFTFMVKDT SYLFITGPDV VKSVTNEDVT
QEELGGAKTH TTMSGVAHRA FENDVDALCN LRDFFNYLPL
SSQDPAPVRE CHDPSDRLVP ELDTIVPLES TKAYNMVDII
HSVVDEREFF EIMPNYAKNI IVGFARMNGR TVGIVGNQPK
VASGCLDINS SVKGARFVRF CDAFNIPLIT FVDVPGFLPG
TAQEYGGIIR HGAKLLYAFA EATVPKVTVI TRKAYGGAYD
VMSSKHLCGD TNYAWPTAEI AVMGAKGAVE IIFKGHENVE
AAQAEYIEKF ANPFPAAVRG FVDDIIQPSS TRARICCDLD
VLASKKVQRP WRKHANIPL
(SEQ ID NO: 5)
MAAALRVAAV GARLSVLASG LRAAVRSLCS QATSVNERIE
NKRRTALLGG GQRRIDAQHK RGKLTARERI SLLLDPGSFV
ESDMFVEHRC ADFGMAADKN KFPGDSVVTG RGRINGRLVY
VFSQQIIGWA QWLPLVISAL WEAEDFTVFG GSLSGAHAQK
ICKIMDQAIT VGAPVIGLND SGGARIQEGV ESLAGYADIF
LRNVTASGVI PQISLIMGPC AGGAVYSPAL TDFTFMVKDT
SYLFITGPDV VKSVTNEDVT QEELGGAKTH TTMSGVAHRA
FENDVDALCN LRDFFNYLPL SSQDPAPVRE CHDPSDRLVP
ELDTIVPLES TKAYNMVDII HSVVDEREFF EIMPNYAKNI
IVGFARMNGR TVGIVGNQPK VASGCLDINS SVKGARFVRF
CDAFNIPLIT FVDVPGFLPG TAQEYGGIIR HGAKLLYAFA
EATVPKVTVI TRKAYGGAYD VMSSKHLCGD TNYAWPTAEI
AVMGAKGAVE IIFKGHENVE AAQAEYIEKF ANPFPAAVRG
FVDDIIQPSS TRARICCDLD VLASKKVQRP WRKHANIPL

An exemplary mRNA sequence encoding mouse PCCA is published as NCBI reference no. NM_144844. An exemplary protein sequence encoding mouse PCCA is published as NCBI reference no. NP_659093 (SEQ ID NO: 6). For a complete summary of mouse PCCA genomic sequence and other information, see NCBI database Gene ID: 110821.

(SEQ ID NO: 6)
MAGQVWRTVA LLAARRHWRR SSQQQLLGTL KHAPVYSYQC
LVVSRSLSSV EYEPKEKTFD KILIANRGEI ACRVIKTCKK
MGIKTVAIHS DVDASSVHVK MADEAVCVGP APTSKSYLNM
DAIMEAIKKT RAQAVHPGYG FLSENKEFAK RLAAEDVTFI
GPDTHAIQAM GDKIESKLLA KRAKVNTIPG FDGVVKDADE
AVRIAREIGY PVMIKASAGG GGKGMRIAWD DEETRDGFRF
SSQEAASSFG DDRLLIEKFI DNPRHIEIQV LGDKHGNALW
LNERECSIQR RNQKVVEEAP SIFLDPETRQ AMGEQAVALA
KAVKYSSAGT VEFLVDSQKN FYFLEMNTRL QVEHPVTECI
TGLDLVQEMI LVAKGYPLRH KQEDIPISGW AVECRVYAED
PYKSFGLPSI GRLSQYQEPI HLPGVRVDSG IQPGSDISIY
YDPMISKLVT YGSDRAEALK RMEDALDNYV IRGVTHNIPL
LREVIINTRF VKGDISTKFL SDVYPDGFKG HTLTLSERNQ
LLAIASSVFV ASQLRAQRFQ EHSRVPVIRP DVAKWELSVK
LHDEDHTVVA SNNGPAFTVE VDGSKLNVTS TWNLASPLLS
VNVDGTQRTV QCLSREAGGN MSIQFLGTVY KVHILTKLAA
ELNKFMLEKV PKDTSSTLCS PMPGVVVAVS VKPGDMVAEG
QEICVIEAMK MQNSMTAGKM GKVKLVHCKA GDTVGEGDLL
VELE

Exemplary mRNA sequences encoding mouse PCCB are published as NCBI reference nos. NM_025835 (isoform 1) and NM_001311149 (isoform 2). Exemplary protein sequences encoding mouse PCCB are published as NCBI reference nos. NP_080111 (isoform 1, SEQ ID NO: 7) and NP_001298078 (isoform 2, SEQ ID NO: 8). For a complete summary of mouse PCCB genomic sequence and other information, see NCBI database Gene ID: 66904.

(SEQ ID NO: 7)
MAAAIRIRAV AAGARLSVLN CGLGITTRGL CSQPVSVKER
IDNKRHAALL GGGQRRIDAQ HKRGKLTARE RISLLLDPGS
FMESDMFVEH RCADFGMAAD KNKFPGDSVV TGRGRINGRL
VYVFSQDFTV FGGSLSGAHA QKICKIMDQA ITVGAPVIGL
NDSGGARIQE GVESLAGYAD IFLRNVTASG VIPQISLIMG
PCAGGAVYSP ALTDFTFMVK DTSYLFITGP EVVKSVTNED
VTQEQLGGAK THTTVSGVAH RAFDNDVDAL CNLREFFNFL
PLSSQDPAPI RECHDPSDRL VPELDTVVPL ESSKAYNMLD
IIHAVIDERE FFEIMPSYAK NIVVGFARMN GRTVGIVGNQ
PNVASGCLDI NSSVKGARFV RFCDAFNIPL ITFVDVPGFL
PGTAQEYGGI IRHGAKLLYA FAEATVPKIT VITRKAYGGA
YDVMSSKHLL GDTNYAWPTA EIAVMGAKGA VEIIFKGHQD
VEAAQAEYVE KFANPFPAAV RGFVDDIIQP SSTRARICCD
LEVLASKKVH RPWRKHANIP L
(SEQ ID NO: 8)
MAAAIRIRAV AAGARLSVLN CGLGITTRGL CSQPVSVKER
IDNKRHAALL GGGQRRIDAQ HKRGKLTARE RISLLLDPGS
FMESDMFVEH RCADFGMAAD KNKFPGDSVV TGRGRINGRL
VYVFSQDFTV FGGSLSGAHA QKICKIMDQA ITVGAPVIGL
NDSGGARIQE GVESLAGYAD IFLDTSYLFI TGPEWKSVT
NEDVTQEQLG GAKTHTTVSG VAHRAFDNDV DALCNLREFF
NFLPLSSQDP APIRECHDPS DRLVPELDTV VPLESSKAYN
MLDIIHAVID EREFFEIMPS YAKNIVVGFA RMNGRTVGIV
GNQPNVASGC LDINSSVKGA RFVRFCDAFN IPLITFVDVP
GFLPGTAQEY GGIIRHGAKL LYAFAEATVP KITVITRKAY
GGAYDVMSSK HLLGDTNYAW PTAEIAVMGA KGAVEIIFKG
HQDVEAAQAE YVEKFANPFP AAVRGFVDDI IQPSSTRARI
CCDLEVLASK KVHRPWRKHA NIPL

In some embodiments, the at least one subunit of human PCC or a biologically active fragment thereof, encoded in full-length or fragment(s) by the mRNA of the instant disclosure comprises at least a protein sequence with at least 70%, 75%, 80%, 85%, 90%, 91%, 92%, 93%, 94%, 95%, 96%, 97%, 98%, 99%, or 100% identity to at least one of SEQ ID NOs: 1-8. In some embodiments, the mRNA of the instant disclosure encoding at least one subunit of human PCC or a biologically active fragment thereof comprises at least a nucleotide sequence with at least 70%, 75%, 80%, 85%, 90%, 91%, 92%, 93%, 94%, 95%, 96%, 97%, 98%, 99%, or 100% identity to a nucleotide sequence which encodes at least one of SEQ ID NOs: 1-8.

The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is known in the art.

Percent identity can be determined using a sequence alignment tool or program, including but not limited to (1) a BLAST 2.0 Basic BLAST homology search using blastp for amino acid searches and blastn for nucleic acid searches with standard default parameters, wherein the query sequence is filtered for low complexity regions by default; (2) a BLAST 2 alignment (using the parameters described below); (3) PSI BLAST with the standard default parameters (Position Specific Iterated BLAST; (4) and/or Clustal Omega. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches.

One of ordinary skill in the art will recognize that individual substitutions, deletions or additions to a nucleic acid, peptide, polypeptide or protein sequences that alter, add or delete a single amino acid or a small percentage of amino acids in the encoded sequence is a “conservatively modified variant.” Such variants can be useful, for example, to alter the physical properties of the peptide, e.g., to increase stability or efficacy of the peptide. Conservative substitution tables providing functionally similar amino acids are known to those of ordinary skill in the art. Such conservatively modified variants are in addition to and do not exclude polymorphic variants, interspecies homologs and alternate alleles. The following groups provide non limiting examples of amino acids that can be conservatively substituted for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (5), Threonine (T); and 8) Cysteine (C), Methionine (M).

The term “codon-optimized” refers to genes or coding regions of a nucleic acid molecule to be translated into a polypeptide sequence. Due to the degeneracy of the genetic code, there are typically more than one triplet codons that cade for a particular amino acid during translation. Some codons are more commonly used to encode a particular amino acid by particular organisms, and translation efficiency can be improved by changing the mRNA sequence in such a way as the desired codons are effectively used by the desired host translation machinery. This process, where the mRNA sequence is changed to reflect alternate codon usage to improve translation efficiency without affecting the sequence of the translated polypeptide, is referred to as “codon optimization.” One of skill in the art will recognize, that several algorithms are available to codon optimize an mRNA sequence in silico. In particular embodiments, the modified mRNA molecules are codon-optimized.

Codon usage bias refers to differences in the frequency of occurrence of synonymous codons in coding DNA (Hershberg, R. & Petrov, D., Annu. Rev. Genet., 42:287-99, 2008; Eyre-Walker, A., J. Mol. Evol., 33:442-9, 1991). A codon is a series of three nucleotides (triplets) that encodes a specific amino acid residue in a polypeptide chain or for the termination of translation (stop codons). There are 64 different codons (61 codons encoding for amino acids plus 3 stop codons) for only 20 different translated amino acids. The overabundance in the number of codons allows many amino acids to be encoded by more than one codon. Different organisms often show particular preferences for one of the several codons that encode the same amino acid. Codon preferences reflect a balance between mutational biases and natural selection for translational optimization. Optimal codon usage in fast growing microorganisms, like Escherichia coli or Saccharomyces cerevisiae, for example, reflects the composition of their respective genomic tRNA pool. Optimal codon usage may help to achieve faster translation rates and high accuracy. As a result of these factors, translational selection is expected to be stronger in highly expressed genes, as is indeed the case for the above-mentioned organisms.

In organisms that do not show high growing rates or that present small genomes, codon usage optimization is normally absent, and codon preferences are determined by the characteristic mutational biases seen in that particular genome. Examples of this are Homo sapiens and Helicobacter pylori. Organisms that show an intermediate level of codon usage optimization include at least Drosophila melanogaster, Caenorhabditis elegans, Strongylocentrotus purpuratus and Arabidopsis thaliana.

The modRNA molecules described herein can comprise at least one codon substituted to create the corresponding biased codon specific to the mammal species for delivering such polynucleotide. One exemplary and non-limiting rationale for this substitution is to decrease host immunogenicity and/or to facilitate protein translation in such mammal species. Alternatively, an mRNA can comprise at least one codon substituted to a non-preferred codon in the host mammal species, as such substitutions allow one of skill in the art to attenuate translation speed and efficiency, e.g., to increase differentiation of the expressed protein and/or to add desired properties to the expressed protein or fragment thereof.

### RNA Formation and Modifications

As used herein, the term “nucleic acid” refers to polymeric biomolecules, e.g., genetic material (e.g., oligonucleotides or polynucleotides comprising DNA or RNA), which include any compound and/or substance that comprise a polymer of nucleotides. These polymers are polynucleotides. Nucleic acids described herein include, for example, RNA or stabilized RNA, e.g., modRNA, encoding a protein or enzyme.

The mRNAs described herein can be natural or recombinant, isolated or chemically synthesized. Such mRNAs can be, for example isolated from in vitro cell cultures or from organisms such as plants or animals in vivo. The mRNAs can be, for example, synthesized or produced in silico.

Described herein are compositions and methods for the manufacture and optimization of mRNA molecules, e.g., modRNAs, through modification of the architecture of mRNA molecules. The disclosure provides, for example, methods for increasing production of a PCC or a biologically active fragment thereof encoded by the mRNA molecules by altering mRNA sequence and/or structure.

The modRNA can comprise, for example, one or more chemical/structural modifications. Such modification(s) can, for example, reduce the innate immune response of a cell into which the mRNA molecule is introduced or any of plurality of other desired effects including, but not limited to: 1) improving the stability of the mRNA molecule; 2) improving the efficiency of protein production; 3) improving intracellular retention and/or the half-life of the mRNA molecules; and/or 4) improving viability of contacted cells. Exemplary modification methods and compositions can be seen in, for example, PCT publication Nos. WO2014081507 and WO2013151664, the entire contents of each of which are hereby incorporated by reference.

Provided herein is a modified mRNA molecule containing a translatable region and one, two or more than two different nucleoside modifications. Nucleoside modifications can include, for example, uniform substitution of a ribonucleoside throughout the modRNA, e.g., incorporation of a modified uracil, cytosine, adenine or guanine at every position where uracil, cytosine, adenine or guanine occurs in the mRNA sequence. Alternatively, modifications can occur at specific sequence positions, and thus the modRNA is discreetly modified. In some embodiments, the modRNA exhibits reduced degradation in a cell into which the mRNA is introduced, relative to a corresponding unmodified mRNA. Two or more linked nucleotides, for example, can be inserted, deleted, duplicated, inverted or randomized in the mRNA molecule without significant chemical modification to the mRNA. The chemical modifications can be located on the sugar moiety of an mRNA molecule described herein. The chemical modifications can be located on the phosphate backbone of the mRNA.

The modRNA molecule(s) described herein can be cyclized or concatemerized, to generate a translation competent molecule to assist interactions, for example, between PABPs and 5′ end binding proteins. Cyclization or concatemerization can be achieved, for example, by 1) chemical, 2) enzymatic and/or 3) ribozyme catalyzed processes. The newly formed 5′/3′ linkage can be intramolecular or intermolecular.

modRNA molecules can be, for example, linked using a functionalized linker molecule. A functionalized saccharide molecule, for example, can be chemically modified to contain multiple chemical reactive groups (SH—, NH2-, N3, etc.) to react with the cognate moiety on a 3′ functionalized mRNA molecule (e.g., a 3′ maleimide ester, 3′ NHS ester, alkynyl, etc.). The number of reactive groups on the modified saccharide can be controlled in a stoichiometric fashion to directly control the stoichiometric ratio of conjugated nucleic acid or mRNA.

The mRNA molecule(s) described herein can be conjugated to other polynucleotides, dyes, intercalating agents (e.g., acridines), cross linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases, alkylating agents, phosphate, amino acids, PEG (e.g., PEG 40K), MPEG, [MPEG]2, radiolabeled markers, enzymes, haptens (e.g., biotin), transport/absorption facilitators (e.g., aspirin, vitamin E, folic acid), synthetic ribonucleases, proteins (e.g., glycoproteins), peptides (e.g., molecules having a specific affinity for a co-ligand), antibodies (e.g., an antibody that binds to a specified cell type such as, for example, a cancer cell, endothelial cell, hepatocyte or bone cell), hormones and hormone receptors, non-peptidic species (such as lipids, lectins, carbohydrates, vitamins, and cofactors), or a drug. Conjugation may result in increased stability and/or half-life and may be particularly useful in targeting the mRNA molecule of the instant disclosure to specific sites in the cell, tissue or organism.

An mRNA molecule described herein can be, for example bi-functional, which means the mRNA molecule has or is capable of two functions, or multi-functional. The multiple functionalities, structural or chemical, can be encoded by the mRNA (e.g., the function may not manifest until the encoded product is translated) or may be a property of the mRNA itself. Similarly, bi-functional mRNA molecules may comprise a function that is covalently or electrostatically associated with the mRNA. Multiple functions may be provided in the context of a complex of a modified RNA and another molecule.

The mRNA molecule can be purified after isolating from a cell, a tissue or an organism or chemically synthesized. The purification process may include, for example, clean up, quality assurance, and quality control. Purification may be performed by methods known in the arts such as, for example, chromatographic methods, e.g., using, for example, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, for example, strong anion exchange HPLC, weak anion exchange HPLC, reverse phase HPLC (RP-HPLC), and hydrophobic interaction HPLC (HIC-HPLC). A purified polynucleotide (e.g., mRNA) is present in a form or setting different from that in which it is found in nature or a form or setting different from that in which it existed prior to subjecting it to a treatment or purification method.

A quality assurance and/or quality control check may be conducted using methods such as, but are not limited to, gel electrophoresis, UV absorbance, or analytical HPLC. In another embodiment, the mRNA molecule may be sequenced by methods including, but not limited to, reverse transcriptase PCR.

In one embodiment, the mRNA molecule is quantified using methods such as, for example, ultraviolet visible spectroscopy (UV/Vis). The mRNA molecule can be analyzed to determine if the mRNA is of proper size or if degradation has occurred. Degradation of the mRNA can be checked by methods such as, for example, agarose gel electrophoresis, HPLC based purification methods (e.g., strong anion exchange HPLC, weak anion exchange HPLC, reverse phase HPLC (RP HPLC), and hydrophobic interaction HPLC (HIC HPLC)), liquid chromatography/mass spectrometry (LCMS), capillary electrophoresis (CE) and capillary gel electrophoresis (CGE).

The described mRNA can comprise at least one structural or chemical modification. The nucleoside that is modified in the mRNA, for example, can be a uridine (U), a cytidine (C), an adenine (A), or guanine (G). The modified nucleoside can be, for example, m⁵C (5-methylcytidine), m⁶A (N6-methyladenosine), s²U (2-thiouridien), ψ (pseudouridine) or Um (2-O-methyluridine). Some exemplary chemical modifications of nucleosides in the mRNA molecule further include, for example, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-methoxyuridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, and N²,N²-dimethyl-6-thio guanosine. In another embodiment, the modifications are independently selected from the group consisting of 5-methylcytosine, 5-methoxyuridine, pseudouridine and 1-methylpseudouridine.

In some embodiments, the modified nucleobase in the mRNA molecule is a modified uracil including, for example, pseudouridine (y), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmo⁵U), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (τcm⁵U), 1-taurinomethyl pseudouridine, 5-taurinomethyl-2-thio uridine (™⁵s2U), 1-taurinomethyl-4-thio pseudouridine, 5-methyl uridine (m⁵U, e.g., having the nucleobase deoxythymine), 1-methyl pseudouridine (m¹ψ), 5-methyl-2-thio uridine (m⁵s2U), 1-methyl-4-thio pseudouridine (m¹s⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), .alpha-thio uridine, 2′-O-methyl uridine (Um), 5,2′-O-dimethyl uridine (m⁵Um), 2′-O-methyl pseudouridine (ψm), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-[3-(1-E-propenylamino) uridine.

In some embodiments, the modified nucleobase is a modified cytosine including, for example, 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴₂Cm), 1-thio cytidine, 2′-F-ara cytidine, 2′-F cytidine, and 2′-OH-ara cytidine.

In some embodiments, the modified nucleobase is a modified adenine including, for example, 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms² m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalylcarbamoyl adenosine (hn⁶A), 2-methylthio-N⁶-hydroxynorvalylcarbamoyl adenosine (ms²hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am), N⁶,N⁶,2′-O-trimethyl adenosine (m⁶₂Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modified nucleobase is a modified guanine including, for example, inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyVV), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²₂G), N^{2,7}-dimethyl guanosine (m^{2,7}G), N², N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m²′⁷Gm), 2′-O-methyl inosine (Im), 1,2′-O-dimethyl inosine (m¹Im), 2′-O-ribosyl guanosine (phosphate) (Gr(p)), 1-thio guanosine, O⁶-methyl guanosine, 2′-F-ara guanosine, and 2′-F guanosine.

The nucleobase of the nucleotide can be independently selected from a purine, a pyrimidine, a purine or pyrimidine analog. For example, the nucleobase can each be independently selected from adenine, cytosine, guanine, uracil or hypoxanthine. The nucleobase can also include, for example, naturally occurring and synthetic derivatives of a base, including, but not limited to, pyrazolo[3,4-d]pyrimidines, 5-methylcytosine (5-me-C), 5-hydroxymethyl cytosine, xanthine, hypoxanthine, 2-amino adenine, 6-methyl and other alkyl derivatives of adenine and guanine, 2-propyl and other alkyl derivatives of adenine and guanine, 2-thio uracil, 2-thio thymine and 2-thio cytosine, 5-propynyl uracil and cytosine, 6-azo uracil, cytosine and thymine, pseudouracil, 4-thio uracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methyl guanine and 7-methyl adenine, 8-aza guanine and 8-aza adenine, deaza guanine, 7-deaza guanine, 3-deaza guanine, deaza adenine, 7-deaza adenine, 3-deaza adenine, pyrazolo[3,4-d]pyrimidine, imidazo[1,5-a]1,3,5 triazinones, 9-deaza purines, imidazo[4,5-d]pyrazines, thiazolo[4,5-d]pyrimidines, pyrazine-2-ones, 1,2,4-triazine, pyridazine; and 1,3,5-triazine. When the nucleotides are depicted using the shorthand A, G, C, T or U, each letter refers to the representative base and/or derivatives thereof, e.g., A includes adenine or adenine analogs, e.g., 7-deaza adenine).

Other modifications include, for example, those in U.S. Pat. No. 8,835,108; U.S. Patent Application Publication No. 20130156849; Tavernier, G. et al., J. Control. Release, 150:238-47, 2011; Anderson, B. et al., Nucleic Acids Res., 39:9329-38, 2011; Kormann, M. et al., Nat. Biotechnol., 29:154-7, 2011; Karikó, K. et al., Mol. Ther., 16:1833-40, 2008; Karikó, K. et al., Immunity, 23:165-75, 2005; and Warren, L. et al., Cell Stem Cell, 7:618-30, 2010; the entire contents of each of which is incorporated herein by reference.

### Compositions

The mRNA of the instant disclosure can be delivered into a host, such as a mammal (e.g., a human), to express a protein of interest (i.e., at least one PCC subunit or a biologically active fragment thereof). The mRNA may comprise at least one of exons of the protein of interest for in vivo expression. Optionally, the mRNA may have at least one of the introns of the protein of interest or another protein to facilitate gene expression. For the encoded PCC subunit(s) or biologically active fragment(s) thereof, different subunit polypeptides or domains of the same or different subunit polypeptides can be expressed from a single mRNA molecule or from two different mRNA molecules (e.g., each chain expressing a different subunit). In latter situation these two mRNA molecules will be co-delivered into the host for in vivo expression and construction of the PCCA/PCCB complex. Optionally, the one or two mRNA molecule may be delivered in conjunction with a polypeptide or protein, or an mRNA encoding such polypeptide or protein, which is capable of facilitating protein expression and/or function of PCC complex in the host.

### Delivery

When formulated in a nanoparticle for delivery, modified mRNA show increased nuclease tolerance and is more effectively taken up by tumor cells after systemic administration (Wang, Y. et al., Mol. Ther., 21:358-67, 2013; the content of which is incorporated by reference herein in its entirety). mRNA can be delivered, for example, by multiple methods to the host organism (PCT publication Nos: WO2013185069, WO2012075040 and WO2011068810, the entire contents of each of which is herein incorporated by reference).

Lipid carrier vehicles can be used to facilitate the delivery of nucleic acids to target cells. Lipid carrier vehicles (e.g., liposomes and lipid-derived nanoparticles (LNPs), such as, for example, the MC3 LNP (Arbutus Biopharma)) are generally useful in a variety of applications in research, industry, and medicine, particularly for their use as transfer vehicles of diagnostic or therapeutic compounds in vivo (Lasic, D., Trends Biotechnol., 16:3-7-21, 1998; Drummond, D. et al., Pharmacol. Rev., 51:691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains.

The liposomal transfer vehicles are prepared to contain the desired nucleic acids for the protein of interest. The process of incorporation of a desired entity (e.g., a nucleic acid such as, for example, an mRNA) into a liposome is referred to as “loading” (Lasic, D. et al., FEBS Lett., 312:255-8, 1992). The liposome-incorporated nucleic acids can be completely or be partially located in the interior space of the liposome, within the bilayer membrane of the liposome, or associated with the exterior surface of the liposome membrane. The incorporation of a nucleic acid into liposomes is referred to herein as “encapsulation,” wherein the nucleic acid is entirely contained within the interior space of the liposome. The purpose of incorporating an mRNA into a transfer vehicle, such as a liposome, is often to protect the nucleic acid from an environment that may contain enzymes or chemicals that degrade nucleic acids and/or systems or receptors that cause the rapid excretion of the nucleic acids. Accordingly, the selected transfer vehicle is capable of enhancing the stability of the mRNA contained therein. The liposome allows the encapsulated mRNA to reach a desired target cell.

As used herein, the term “target cell” refers to a cell or tissue to which a composition described herein is to be directed or targeted. In some embodiments, the target cells are deficient in a protein or enzyme of interest. For example, where it is desired to deliver a nucleic acid to a hepatocyte, the hepatocyte represents the target cell. In some embodiments, the nucleic acids and compositions specifically transfect the target cells (i.e., they do not transfect non-target cells). The compositions and methods can be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions described herein can be administered and dosed in accordance with current medical practice, taking into account, for example, the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient expression of the antibody or fragment in the target cell.

The route of delivery used in the methods of the disclosure allows for noninvasive, self-administration of the therapeutic compositions of mRNA described herein. The methods involve intratracheal or pulmonary administration by aerosolization, nebulization, or instillation of compositions comprising the mRNA in a suitable transfection or lipid carrier vehicles as described herein.

Following administration of the composition to the subject, the protein of interest, e.g., PCCA and/or PCCB or biologically active fragment(s) thereof encoded by the mRNA, is detectable in the target tissues for at least about one to about seven days or longer following administration of the composition to the subject. The amount of expressed protein or protein fragment necessary to achieve a therapeutic effect varies depending on the condition being treated and the condition of the patient. The expressed PCC or fragment(s), for example, is detectable in the target tissues at a concentration of at least 0.025-1.5 μg/mL (e.g., at least 0.050 μg/mL, at least 0.075 μg/mL, at least 0.1 μg/mL, at least 0.2 μg/mL, at least 0.3 μg/mL, at least 0.4 μg/mL, at least 0.5 μg/mL, at least 0.6 μg/mL, at least 0.7 μg/mL, at least 0.8 μg/mL, at least 0.9 μg/mL, at least 1.0 μg/mL, at least 1.1 μg/mL, at least 1.2 μg/mL, at least 1.3 μg/mL, at least 1.4 μg/mL, or at least 1.5 μg/mL), or at a higher concentration, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or 45 days or longer following administration of the composition to the subject.

### Pharmaceutical Compositions and Formulations

The mRNA compositions described herein can be formulated as a pharmaceutical solution, e.g., for administration to a subject for the treatment or prevention of a disease or disorder associated with PCC deficiency, e.g., PA. The pharmaceutical compositions can include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (Berge, S. et al., J. Pharm. Sci., 66:1-19, 1977).

The compositions can be formulated according to methods in the art (Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7th Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). A composition can be formulated, for example, as a buffered solution at a suitable concentration and suitable for storage at 2-8 C (e.g., 4 C). In some embodiments, a composition can be formulated for storage at a temperature below 0 C (e.g., −20 C or −80 C). In some embodiments, the composition can be formulated for storage for up to two years (e.g., one month, two months, three months, four months, five months, six months, seven months, eight months, nine months, 10 months, 11 months, 1 year, 1% years or 2 years). Thus, in some embodiments, the compositions described herein are stable in storage for at least one year at 2-8 C (e.g., 4 C).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing an mRNA molecule intended for systemic or local delivery can be in the form of injectable or infusible solutions. Accordingly, the compositions can be formulated for administration by a parenteral mode (e.g., intravenous, subcutaneous, intraperitoneal or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

The compositions can be formulated as a solution, microemulsion, dispersion, liposome or other ordered structure suitable for stable storage at high concentration. Sterile injectable solutions can be prepared by incorporating a composition described herein in the required amount in an appropriate solvent with one or a combination of ingredients enumerated above, as required or otherwise desirable, followed by filter sterilization. Dispersions are generally prepared by incorporating a composition into a sterile vehicle that contains a basic dispersion medium and other ingredients from those enumerated above. In the case of sterile powders for the preparation of sterile injectable solutions, methods for preparation include vacuum drying and freeze-drying that yield a powder of a composition plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts and gelatin.

The mRNA compositions described herein can also be formulated in liposome compositions prepared by methods known in the art (e.g., Eppstein, D. et al., Proc. Natl. Acad. Sci. USA, 82:3688-92, 1985; Hwang, K. et al., Proc. Natl. Acad. Sci. USA, 77:4030-4, 1980; and U.S. Pat. Nos. 4,485,045; 4,544,545 and 5,013,556; the entire contents of each of which is incorporated by reference herein).

Compositions can be formulated with a carrier, for example, which protects the formulated mRNA against rapid release, such as a controlled release formulation, including implants and microencapsulated delivery systems. Biodegradable, biocompatible polymers, for example, can be used (e.g., ethylene vinyl acetate, polyanhydrides, polyglycolic acid, collagen, polyorthoesters and polylactic acid). Many methods for the preparation of such formulations are known in the art (e.g., J. R. Robinson (1978) “Sustained and Controlled Release Drug Delivery Systems,” Marcel Dekker, Inc., New York).

Compositions can be formulated for delivery to the eye. As used herein, the term “eye” refers to any and all anatomical tissues and structures associated with an eye.

In some embodiments, compositions can be administered locally, for example, by way of topical application or intravitreal injection. For example, in some embodiments, the compositions can be formulated for administration by way of an eye drop.

The therapeutic preparation for treating the eye can contain one or more active agents in a concentration from about 0.01 to about 1% by weight, preferably from about 0.05 to about 0.5% in a pharmaceutically acceptable solution, suspension or ointment. The preparation can be, for example, in the form of a sterile aqueous solution containing, e.g., additional ingredients such as, but are not limited to, preservatives, buffers, tonicity agents, antioxidants and stabilizers, nonionic wetting or clarifying agents and viscosity-increasing agents.

Suitable preservatives for use in such a solution include, for example, benzalkonium chloride, benzethonium chloride, chlorobutanol, thimerosal and the like. Suitable buffers include, e.g., boric acid, sodium and potassium bicarbonate, sodium and potassium borates, sodium and potassium carbonate, sodium acetate, and sodium biphosphate, in amounts sufficient to maintain the pH at between about pH 6 and about pH 8, and preferably, between pH 7 and pH 7.5. Suitable tonicity agents include, for example, dextran 40, dextran 70, dextrose, glycerin, potassium chloride, propylene glycol and sodium chloride.

Suitable antioxidants and stabilizers include, for example, sodium bisulfite, sodium metabisulfite, sodium thiosulfite and thiourea. Suitable wetting and clarifying agents include, for example, polysorbate 80, polysorbate 20, poloxamer 282 and tyloxapol. Suitable viscosity-increasing agents include, for example, dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone and carboxymethylcellulose.

As described above, relatively high concentration (mRNA) compositions can be made. For example, the compositions can be formulated at an mRNA concentration between about 10 mg/mL to about 100 mg/mL (e.g., between about 9 mg/mL and about 90 mg/mL; between about 9 mg/mL and about 50 mg/mL; between about 10 mg/mL and about 50 mg/mL; between about 15 mg/mL and about 50 mg/mL; between about 15 mg/mL and about 110 mg/mL; between about 15 mg/mL and about 100 mg/mL; between about 20 mg/mL and about 100 mg/mL; between about 20 mg/mL and about 80 mg/mL; between about 25 mg/mL and about 100 mg/mL; between about 25 mg/mL and about 85 mg/mL; between about 20 mg/mL and about 50 mg/mL; between about 25 mg/mL and about 50 mg/mL; between about 30 mg/mL and about 100 mg/mL; between about 30 mg/mL and about 50 mg/mL; between about 40 mg/mL and about 100 mg/mL; or between about 50 mg/mL and about 100 mg/mL). In some embodiments, compositions can be formulated at a concentration of greater than 5 mg/mL and less than 50 mg/mL. Methods for formulating a protein in an aqueous solution are known in the art, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (2005), “Therapeutic peptides and proteins: formulation, processing, and delivery systems, Second Edition” CRC Press.

In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9 or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8 or 7.9), but less than pH 8.

In some embodiments, compositions can be formulated with one or more additional therapeutic agents, e.g., additional therapies for treating or preventing a disease or disorder described herein, e.g., PCC-deficiency-associated disease or disorder in a subject. When compositions are to be used in combination with a second active agent, the compositions can be co-formulated with the second agent or the compositions can be formulated separately from the second agent formulation. The respective pharmaceutical compositions can be mixed, for example, just prior to administration, and administered together or can be administered separately, e.g., at the same or different times.

### EXAMPLES

### Example 1 Materials and Methods

### Cell Lines and Culture Media

HepG2, Hep3B, SNU-475, HeLa, NIH-3T3, HEK293, Calu-3, H2291, H522 and HPAF-II were purchased from ATCC (Manassas, Va.) and maintained according to provider's instructions. Patient-derived lymphoblastoid cells (LCLs) and fibroblasts were obtained from Coriell Biorepository (Camden, N.J.) and maintained according to provider's instructions. Primary mouse hepatocytes were purchased from Triangle Research Laboratories (Durham, N.C.) and maintained according to provider's instructions.

HeLa, HepG2, Hep3B, Calu3, HPAFII, H2291 and HEK293s were maintained in Eagle's MEM (Corning, Manassas, Va.) supplemented with 10% heat-inactivated fetal bovine serum (Tissue Culture Biologicals, Long Beach, Calif.)) and 2 mM L-glutamine (Corning, Manassas, Va.). H522 and SNU-475 were maintained in RPMI-1640 (Corning, Manassas, Va.) supplemented with 10% heat-inactivated fetal bovine serum Tissue Culture Biologicals, Long Beach, Calif.) and 2 mM L-glutamine (Corning, Manassas, Va.). NIH-3T3s were maintained in DMEM (Corning, Manassas, Va.) supplemented with 10% heat-inactivated fetal bovine serum (Tissue Culture Biologicals, Long Beach, Calif.) and 2 mM L-glutamine (Corning, Manassas, Va.). Fibroblasts were maintained in DMEM (Corning, Manassas, Va.) supplemented with 20% heat-inactivated fetal bovine serum (Tissue Culture Biologicals, Long Beach, Calif.) and 2 mM L-glutamine (Corning, Manassas, Va.). LCLs were maintained in RPMI (Corning, Manassas, Va.) supplemented with 15% heat-inactivated fetal bovine serum Tissue Culture Biologicals, Long Beach, Calif.) and 2 mM L-glutamine(Corning, Manassas, Va.). DPBS was purchased from Corning (Manassas, Va.). Primary mouse liver hepatocytes were plated in animal hepatocyte plating media (Triangle Research labs, Durham, N.C.) and maintained in hepatocyte maintenance media (Triangle Research labs, Durham, N.C.)

Chemical reagents used for mitochondrial isolation were purchased from Sigma (St. Louis, Mo.).

### Antibodies (for Western Blot and IF)

Antibodies used include Rabbit anti-PCCA (Cat No. 21988-1-AP, ProteinTech, Chicago, Ill.), Rabbit anti-PCCB (Cat No. NBP1-85886, Novus Biologicals, Littleton, Colo.), Rabbit anti-GAPDH (Cell Signaling Technologies, Danvers, Mass.), Rabbit anti-FLAG (Cell Signaling Technologies, Danvers, Mass.), mouse anti-vinculin (Sigma, St. Louis, Mo.), and Rabbit anti-COXIV (Cell Signaling Technologies, Danvers, Mass.).

### DNA Plasmids

pCMV6-XL5 (Cat No. PCMV6XL5) and pCMV-hPCCA(untagged) (Cat No. SC120017) were purchased from Origene (Rockville, Md.).

qRT-PCR Primers

Gene expression was performed using Gene Expression Master Mix or Taqman Fast Advanced Master Mix (Life Technologies, Carlsbad, Calif.) according to manufacturer's protocol. The following Taqman assays (Life Technologies, Carlsbad, Calif.) were used to measure mRNA expression discussed hereafter: human PCCA (Hs00165407_m1), human PCCB (Hs00166909_m1), human GAPDH (Hs03929097_g1), mouse PCCA (Mm00454899_m1), Mouse beta Actin (Cat No. 4352341E).

DNA Oligo primers used for modRNA-specific transcript were synthesized at Integrated DNA Technologies (Coralville, Iowa), including: mPCCA01 modRNA_4F (5′-TGGGAAAATGGGCAAGGTGA-3′; SEQ ID NO:9) and mPCCA01 modRNA 4R (5′-ACCGAGGCTCCAGCCTATTA-3′; SEQ ID NO:10), and measured using PowerSYBR Master Mix (Life Technologies, Carlsbad, Calif.) according to manufacturer's protocol.

### DNA/modRNA Transfection Protocol

DNA transfection in HepG2 cells was performed using TransfeX™ Transfection Reagent (ATCC, Manassas, Va.) according to manufacturer instructions. DNA transfection in H522, Hep3B, SNU-475, and HeLa cells were performed using Lipofectamine® 3000 (Life Technologies, Carlsbad, Calif.) according to manufacturer instructions. Cells were transfected with pCMV-PCCA(untagged) DNA construct or pCMV6-XL5 empty vector. Lipid:DNA complexes were incubated in Opti-Mem Reduced Serum media (Life Technologies, Carlsbad, Calif.) and added to culture media. Cells were incubated with DNA:Lipid complex for 6 hours. Cells were then washed once with dPBS and given fresh maintenance media. DNA was transfected into patient LCLs and fibroblasts using Amaxa 4D-Nucleofector System (Lonza, Basel, Switzerland) according to manufacturer instructions.

For transfection of modRNA, patient fibroblasts and primary mouse hepatocytes were transfected with PCCA modRNA using Lipofectamine MessengerMax™ (Life Technologies, Carlsbad, Calif.) according to manufacturer's instructions. After six hours post-transfection, cells were washed once with DPBS and given fresh maintenance media as described in Example 1. Cells transfected with luciferase or eGFP modRNA was used as negative controls.

### Western Blot

PCCA and PCCB protein expression was measured by standard chemiluminscence-based or infrared fluorescence-based Western blot methods. Images were acquired using FluorChemo R system (ProteinSimple, San Jose, Calif.) or Odyssey CLx instrument (Li-Cor, Lincoln, Nebr.).

### PCC Enzyme Assay Method

PCC enzyme activity was measured using ¹⁴C-based radiochemical assay (Weyler, W. et al., Clin. Chim. Acta., 76:321-8, 1977) and performed at UCSD Biochemical Genetics Laboratory (San Diego, Calif.).

qRT-PCR Protocol

mRNA was isolated from cells or tissue using the RNeasy Mini Kit (Qiagen, Germantown, Md.) according to manufacturer instructions. 250 ng-1 μg mRNA was reverse transcribed using High Capacity cDNA using the High Capacity Reverse Transcription Kit (Life Technologies, Carlsbad, Calif.). 10-100 ng of synthesized cDNA was amplified using Taqman-based or SYBR-green based methods according to manufacturer's instructions. QuantStudio 7 (Life Technologies, Carlsbad, Calif.) was used for data acquisition and analysis.

### Liver Mitochondria Preparation

Mouse livers were homogenized in IBc buffer (10 mM Tris-MOPS, 1 mM EGTA/Tris, 200 mM sucrose) supplemented with protease cocktail inhibitor. An aliquot of crude liver homogenate lysate was saved, while the rest of the samples were used for mitochondrial fraction enrichment through sequential centrifugation. Supernatant resulting from the centrifugation of crude lysate at low speed (600×g for 10 minutes, 2 times) was then subjected for high speed centrifugation (7000×g for 10 minutes, 2 times). The resulting mitochondria pellet is used for western blot and PCC enzyme activity analyses.

### Example 2 In Vitro Overexpression of PCCA DNA

Endogenous PCCA and PCCB mRNA and protein expression levels were analyzed in multiple immortalized cell types. Specifically, immortalized cells were harvested from 10 cm plate in RIPA buffer (containing phosphatase/protease inhibitors). Protein lysate was prepared by sonication at 4 C followed by centrifugation at 15,000 rpm for 15 min at 4 C. Gene expression for PCCA and PCCB was measured by qPCR analysis. Protein levels were detected via western blot analysis performed as described in Example 1.

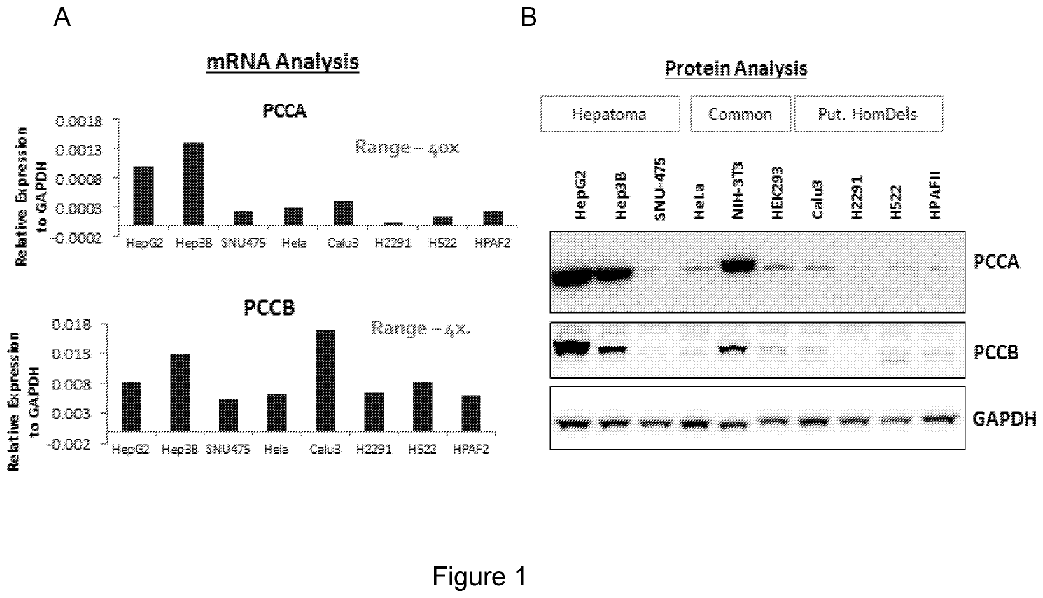

As shown in FIG. 1, mRNA and protein expression levels of PCCA and PCCB showed considerable variability among cell lines. PCCB mRNA was expressed in excess to PCCA mRNA and with less variability among cell lines. However, PCCA and PCCB protein levels were directly correlated, suggesting that PCCB protein stabilization is dependent on PCCA.

PCCA and PCCB protein levels were further tested in PCCA-deficient patient lymphoblastoid cell lines (LCLs) and fibroblasts. Specifically, 10 human lymphoblastoid cell lines (LCLs) and 9 human fibroblasts collected from healthy human, PA patients, and PA gene carriers (parent of patients) were obtained from Coriell Institute for Medical Research (Camden, N.J.). While the genotypes of LCLs were readily available from Coriell, the mutations in PCCA and PCCB in the fibroblasts were discovered by genotyping performed at Emory Genetics Lab (Decatur, Ga.). Mutations in PCCA and PCCB in patient cells include frameshift, nonsense, missense, intron skipping, short sequence deletion and duplication. To characterize PCCA and PCCB protein levels in patient-derived cells, cell lysates were prepared and PCCA, PCCB and GAPDH were detected by western Blot analysis as described in Example 1.

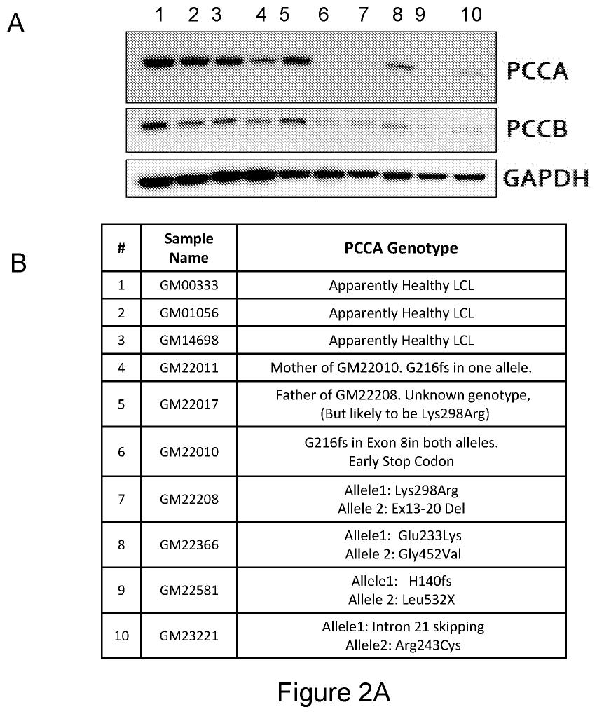

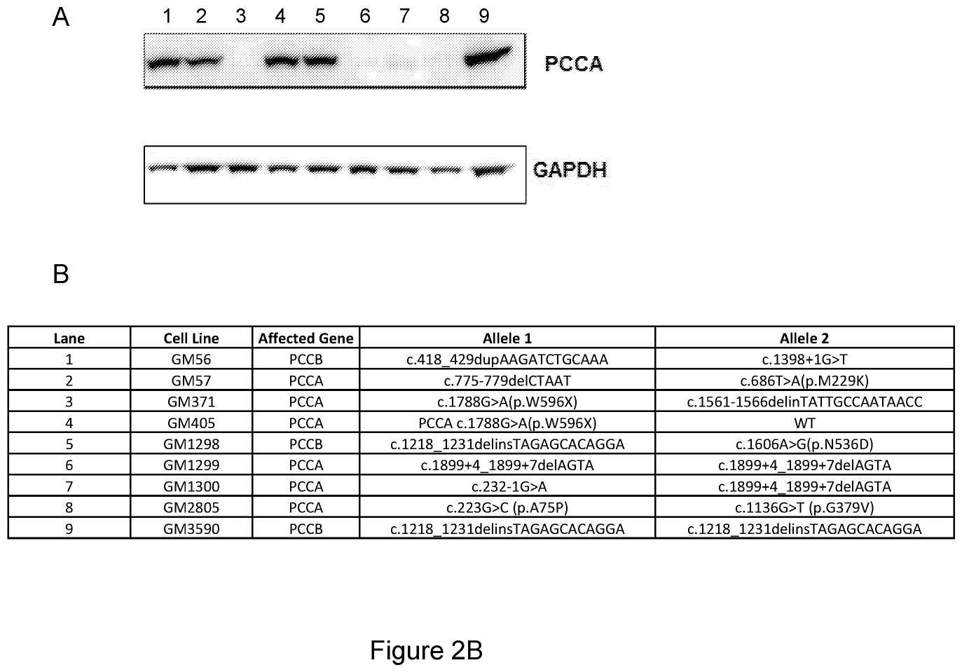

As shown in FIG. 2A, PCCA protein expression levels were dramatically reduced in all five patient LCLs (near none in GM22010 and GM22581). Clinically unaffected parents of PA patients carry PCCA mutations in only one allele, explaining that the PCCA levels of the parents fell between healthy donors and their patient children. In cells with only PCCA mutations (e.g., GM22010 and GM22581), PCCB levels were very well correlated with PCCA levels. On the contrary, PCCA levels were independent of PCCB levels in patients with PCCB mutations (e.g., GM56, GM1298, and GM3590 as in FIG. 3A). This again suggests that PCCB subunit is rapidly turned over in the absence of PCCA, while PCCA can be stable by itself. Comparing FIG. 2A and FIG. 3A, the near absence of expression of PCCA in exemplary cell lines with homozygous or compound heterozygous frameshift and nonsense mutations (e.g., in GM22010 and GM22581) may be likely due to introduction of early stop codon. Missense mutations had variable protein levels (low in GM22366 and high in GM57), which can be explained by the different impact of point mutations on protein stability.

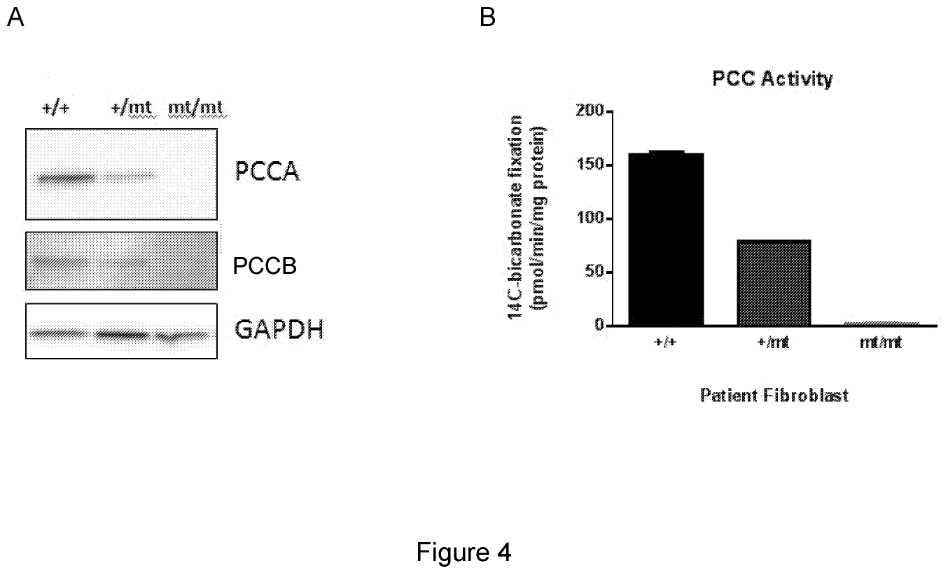

PCC activity was also found to be reduced in PA patient fibroblasts (FIG. 4). Specifically, cell lysate was prepared from normal human dermal fibroblasts (NHDF, shown as “+/+”), PA patient fibroblasts (GM371, shown as “mt/mt”), and clinically unaffected father of GM371 (GM405, shown as “+/mt”)). PCCA and PCCB protein levels were detected via western blot analysis with GAPDH as the loading control. Cells were also harvested and shipped to UCSD Biochemical Genetics Lab (La Jolla, Calif.) to measure PCC enzyme activity. The assay was performed as described in Example 1.

As shown in FIG. 4, The PCC activity and PCCA/B protein levels show gene-dosage dependent manner in fibroblasts. PCC activity detected in the parent fibroblasts (GM405, as “+/mt”) was approximately half of that in normal fibroblasts, while very low activity was detected in PA patient fibroblasts (GM371, as “mt/mt”) (FIG. 4B). The activities correlated very well with PCCA and PCCB protein levels, confirming the deficient PCC protein level and enzyme activity in PA patients.

PCCA/B levels in immortalized cells were analyzed after transfection of PCCA DNA. About 1.5 to 2 million of different immortalized cells were seeded in 60 mm plates and grown for 1 day in 5 mL medium, and then transfected with control and PCCA DNA plasmids using Lipofectamine® 3000 for 6 hours before medium change. After 2 days, cells were harvested and cell lysate was prepared as before. PCCA and PCCB protein levels were detected by western blot.

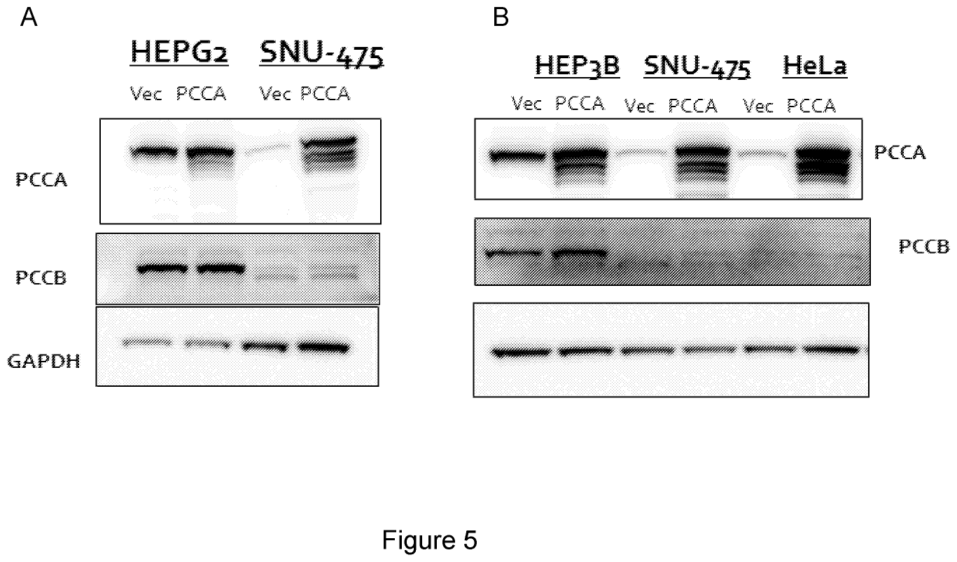

As shown in FIG. 5, in cells with high endogenous PCCA and PCCB level (e.g., HEPG2 and HEP3B), PCCA overexpression did increase the PCCA protein level but merely marginally. Marked increase in PCCA level was detected in cells with low endogenous PCC levels (e.g., SNU-475 and HeLa). In both case, marginal increase in PCCB level can be observed, indicating that PCCA overexpression (by transfection of PCCA DNA plasmids) may stabilize PCCB in immortalized cells. Such stabilization of PCCB by overexpression of PCCA may be crucial for restoring PCC activity, since functional PCC requires both PCCA and PCCB subunit to form a dodecamer complex.

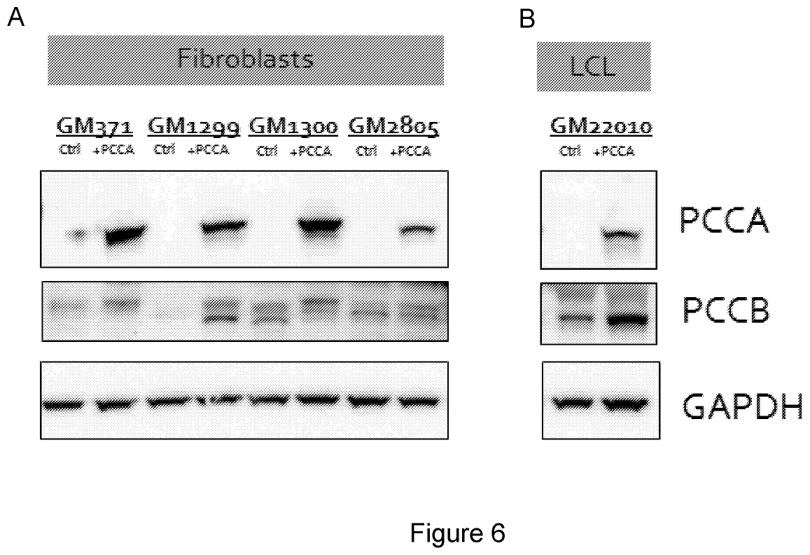

Similarly, patient fibroblasts and lymphoblastoid cells were transfected with PCCA DNA plasmids to overexpress PCCA proteins. Specifically, PCCA-deficient patient fibroblasts (GM371, GM1299, GM1300, GM2805) were nucleofected with empty vector (shown as “ctrl”) or PCCA DNA plasmid (shown as “+PCCA”) as described previously. Cells were harvested for analysis at 24 hour. PA patient LCL (GM22010) was nucleofected with empty vector or PCCA DNA plasmid. Due to the high rate of cell death post nucleofection, LCLs was harvested at 24 hour and dead cells were removed with ficoll gradient centrifugation prior to lysate generation. As shown in FIG. 6, compared to empty vector controls, transfection of PCCA DNA plasmid drastically increased PCCA protein level. Increased PCCB level was also observed, indicating stabilization of PCCB likely through formation of PCC complex with PCCA.

In conclusion, PCCA overexpression by DNA transfection dramatically increased PCCA protein levels mostly in some immortalized (e.g., SNU-475) or PA patient-derived cells (e.g., GM1299 and GM22010). The SNU-475 system is useful to enable evaluation in a liver-specific context. PCCA overexpression in PCCA-deficient cells also increased endogenous PCCB protein levels (probably through stabilization).

### Example 3 In Vitro Overexpression of PCCA mRNA

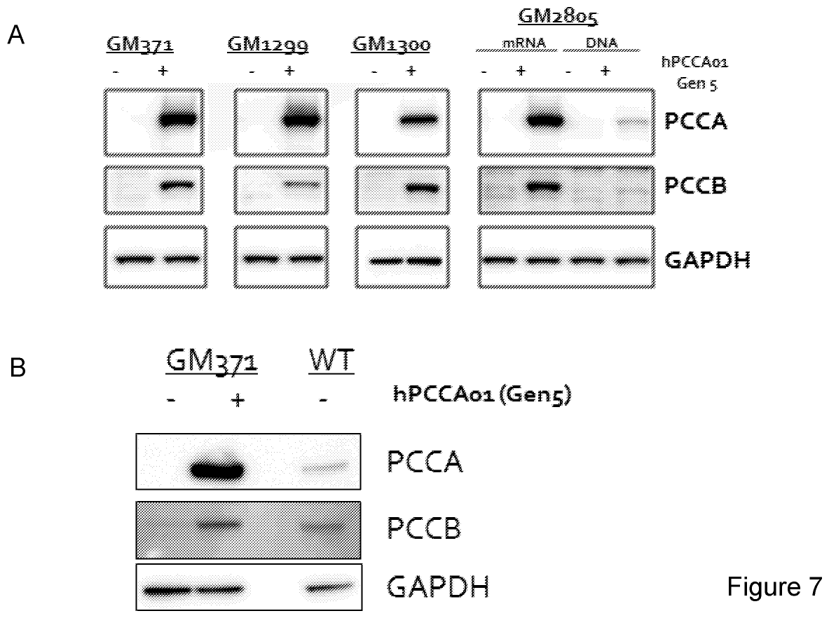

PCCA mRNA (or modRNA) was used to restore PCCA expression in PA patient fibroblasts. Specifically, patient fibroblasts were transfected with either lipid alone as the control or with modRNA encoding human untagged PCCA (hPCCA01) using Lipofectamine® MessengerMax Reagent as described in Example 1, or with PCCA DNA plasmid as described previously. 24 hours after transfection, cells were harvested and cell lysate was prepared. PCCA and PCCB protein levels were detected by western blot. As shown in FIG. 7, transfection of 4 different patient fibroblasts (GM371, GM1299, GM1300 and GM2805) with PCCA modRNA dramatically increased PCCA expression and restored PCCB level above WT level, suggesting successful assembly of PCC complex. Compared to DNA plasmid, much higher expression of PCCA and PCCB was achieved with modRNA transfection. This may be explained by high transfection efficiency of modRNA.

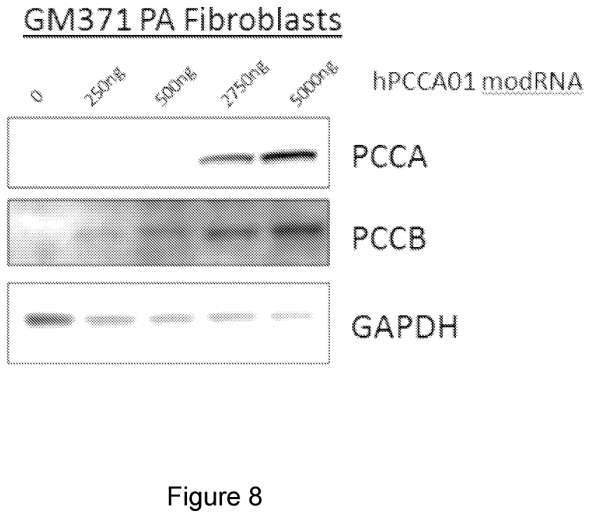

PCCA expression and PCCB stabilization was found to be dependent on modRNA dose. Specifically, GM371 cells were transfected for 24 hour with 0, 250 ng, 1000 ng, 2750 ng or 5000 ng of modRNA hPCCA01, using Lipofectamine® MessengerMax. Cells were harvested at 24 hour after transfection. As shown in FIG. 8, higher modRNA dosages led to higher PCCA and PCCB protein levels.

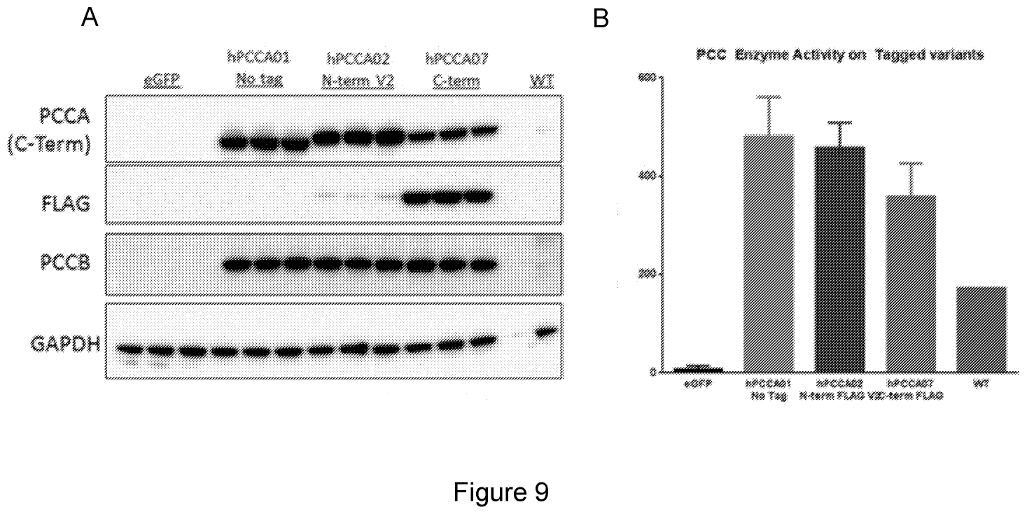

More human PCCA and its FLAG-tagged variant modRNA constructs were prepared and transfected into PCCA-deficient patient fibroblasts. Specifically, modRNAs encoding either N- or C-terminal FLAG-tagged PCCA were synthesized to facilitate distinction of modRNA-expressed proteins from endogenous PCCA. PCCA has a mitochondrial target sequence (MTS) that helps transport the newly synthesized polypeptide to the mitochondria and gets cleaved off upon arrival. For N-terminal FLAG-tagged PCCA, the FLAG sequence was inserted after the putative MTS cleavage site. GM371 cells were transfected for 24 hr with modRNA encoding untagged hPCCA (hPCCA01), hPCCA with N-terminal FLAG tag (hPCCA02), or hPCCA with C-terminal FLAG tag (hPCCA07). At 48 hr, cells were harvested and cell lysates were prepared for western blot analysis of PCCA, PCCB, FLAG and GAPDH. Cell pellet was also frozen down and shipped to UCSD biochemical genetics lab for measurement of PCC enzyme activity. As shown in FIG. 9, PCCA level was dramatically increased with transfection of all three variants, accompanied by restoration of PCCB protein level. Interestingly, FLAG signal was well recognized by anti-FLAG antibodies for C-terminally tagged modRNA variant, while the FLAG signal was much lower for the N-terminal tag variant. This discrepancy may be due to inaccurate prediction of the cleavage site for MTS, which results in FLAG tag not being exposed terminally or cleave-off of the tag.

In agreement with the higher than WT levels of PCCA and PCCB protein, PCC enzyme activity was restored to ˜2 fold or higher of the WT activity (FIG. 9B). The difference in PCC activity between the three variants was not statistically significant, suggesting that the FLAG tag at either terminus did not compromise the enzyme activity. In conclusion, PCCA and it FLAG-tagged variant modRNAs restored PCCA/B expression, and reconstituted PCC activity in PCCA-deficient patient fibroblasts.

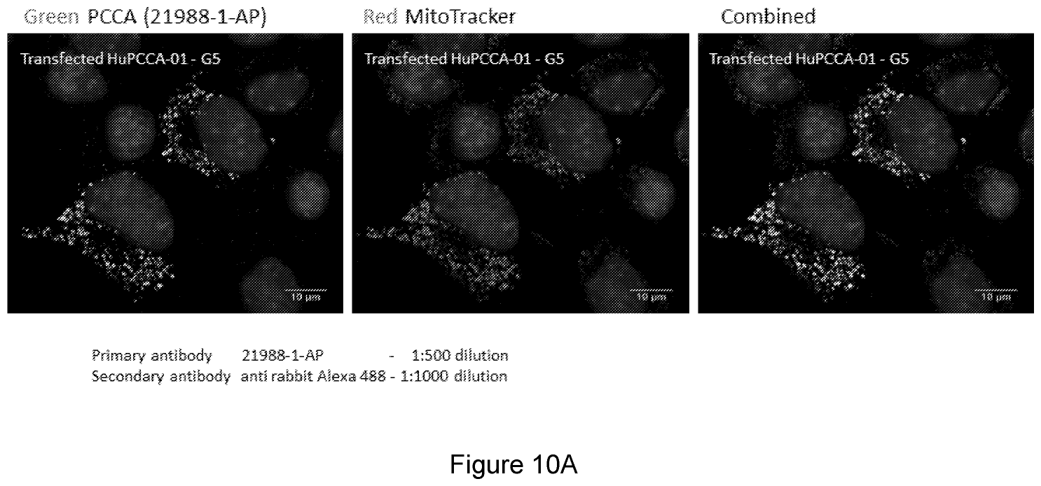

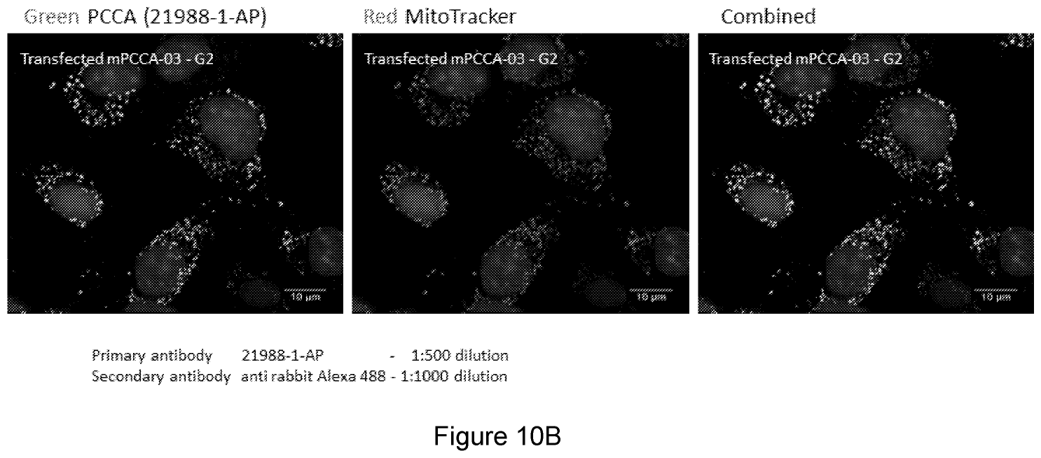

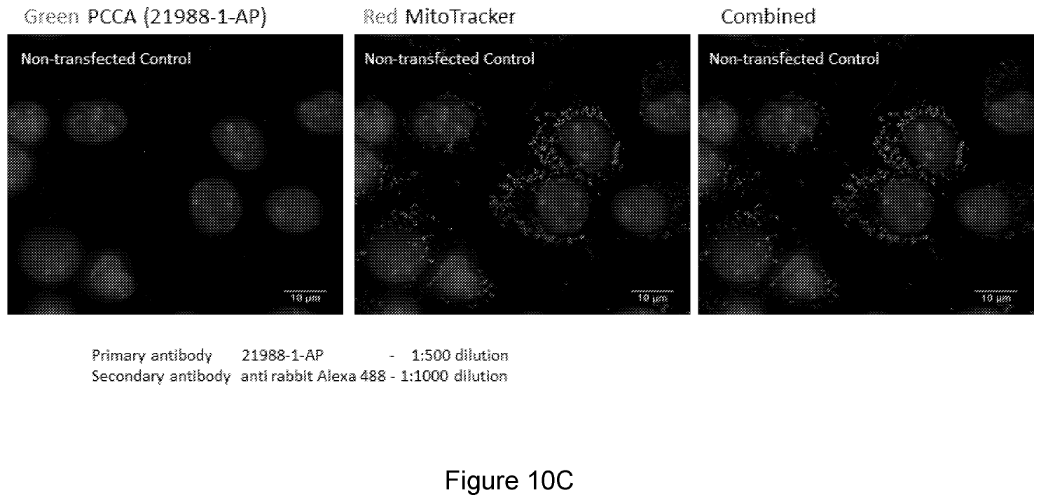

Endogenous PCC is located in the matrix of mitochondria, where the conversion of its substrate Propionyl-CoA to Methylmalonyl-CoA occurs. To study whether PCCA proteins expressed from modRNA were correctly localized to their site of function, localization study with immunofluorescence was performed. Specifically, Hepa1-6 cells with low endogenous PCCA and PCCB level were used. As shown in FIG. 10C, in non-transfected control cells, mitochondria stained with MitoTracker® appeared as a reticulum or as multiple individual punctate organelles. No PCCA signals can be detected in non-transfected cells. In cells transfected with either human or mouse PCCA modRNA, co-localization of PCCA signal (green) with the Mitotracker® signal (red) was observed (FIGS. 10A and 10B), demonstrating that PCCA proteins expressed from modRNA were efficiently targeted into mitochondria after translation.

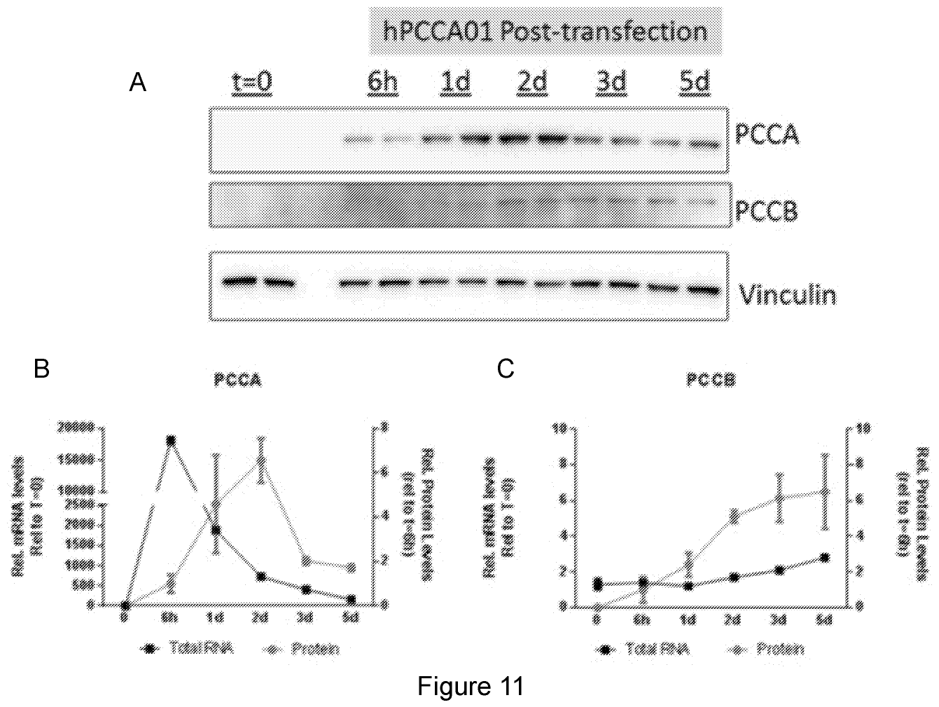

Interestingly, PCCA and PCCB expressions were sustained for five days post transfection of PCCA modRNA. Specifically, PCCA and PCCB protein and mRNA levels were measured by qRT-PCR and western blot analyses. As shown in FIG. 11B, PCCA transcript levels were at a maximum at six hours after transfection and gradually decay over the course of five days, returning to baseline levels by day 5. PCCA protein levels, however, reached maximal expression at two days after transfection (FIGS. 11A and 11B). PCCB mRNA levels showed minor variations over the course of five days after transfection (FIG. 11C), while PCCB protein gradually increased over time and remains stable from day 2 to day 5 after transfection (FIGS. 11A and 11C). Without being limited to this particular theory, the increase in PCCB protein level is likely due to protein stabilization through interactions with modRNA-derived PCCA protein.

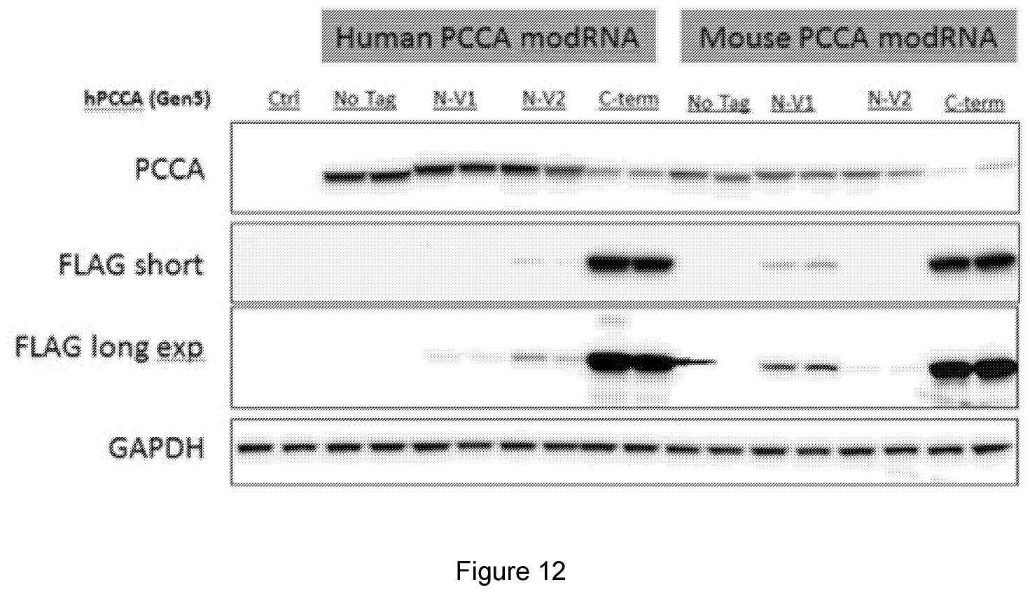

Various PCCA modRNA constructs were used to overexpress PCCA in patient fibroblasts. As shown in FIG. 12, all constructs (untagged PCCA, two N-terminal FLAG-tagged PCCA variants, and C-terminal FLAG-tagged PCCA) dramatically increased PCCA expression. Interestingly, the FLAG antibody detected the C-terminal FLAG tag better than the N-terminal FLAG tag, while the overall protein expression levels of C-terminal FLAG-tagged PCCA was lower than that of the N-terminal tagged variants, according to the anti-PCCA antibody detection. Mouse PCCA showed generally lower expression than human PCCA in patient fibroblasts.

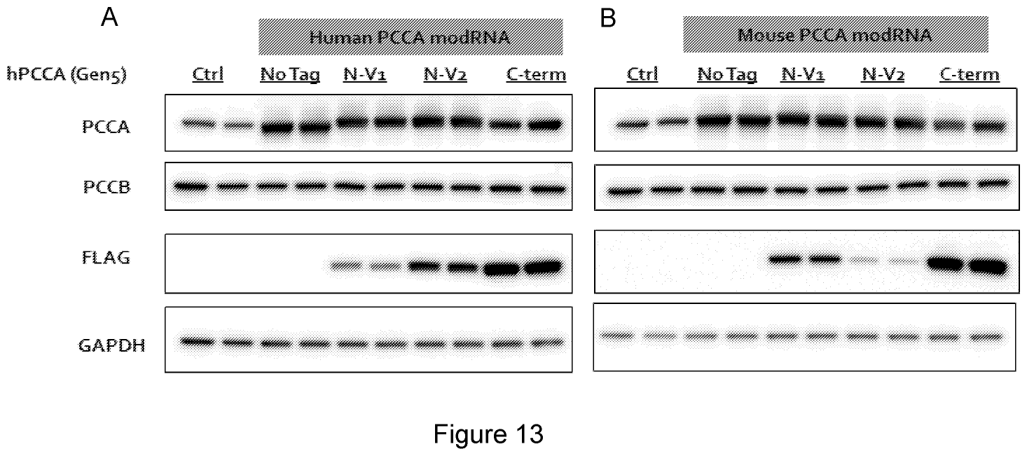

Similarly, human and mouse modRNA constructs were transfected in normal primary mouse liver hepatocytes. The variants include untagged PCCA, two N-terminal FLAG-tagged PCCA variants, and one C-terminal FLAG-tagged PCCA. As shown in FIGS. 13A and 13B, increased PCCA level was detected for all eight variants 24 hour after transfection, while no change in PCCB level was observed. This suggests overexpression of only PCCA subunit was not able to reconstitute more PCC complex in wild-type hepatocytes. As observed before, C-terminal FLAG tag was better recognized by the anti-FLAG antibody than the N-terminal FLAG tag.

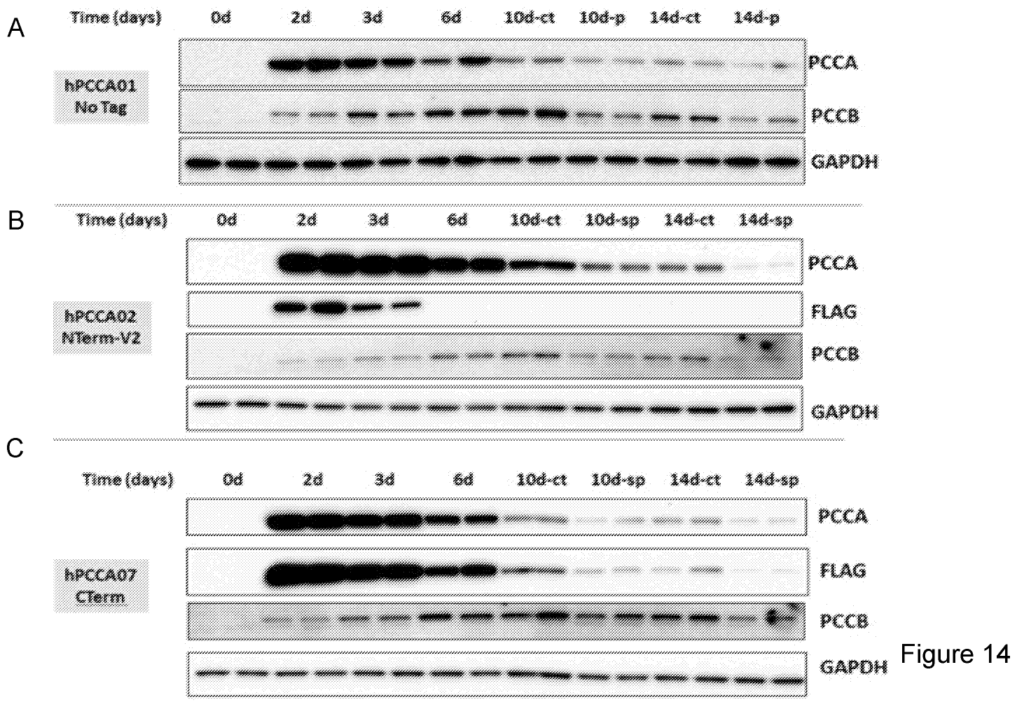

To study the stability of PCC complex post transfection of modRNA, GM371 cells were transfected for 24 hr with human PCCA modRNA variants. Cells were harvested at 0, 2, 3, 6, 10, 14 days after transfection for western blot analysis. To test whether cell proliferation affects modRNA-expressed protein level and stability, on day 6, half of harvested cells were replated at ˜70% confluent and marked as “p” or “sp” to distinguish from continuous culture marked as “ct”. Surprisingly, PCCA and PCCB protein levels were detectable for up to 14 days post-transfection. As shown in FIG. 14, PCCA protein levels peaked at Day 2 post transfection, while PCCB protein levels peaked sometime between Day 6 and Day 10. This different time-course profile suggested a steady accumulation of stable PCC complex that may result from both continuous stabilization of PCCB by expressed PCCA and long half-life of the assembled complex. C-FLAG PCCA was well recognized by anti-FLAG antibody, which the FLAG signal correlated well with the PCCA signal detected by anti-PCCA antibody, demonstrating that the tag is not cleaved off over time. Further, the PCCA/PCCB protein levels were lower in the samples that were split at Day 6 when the same amount of total proteins was loaded for western blot analysis. That is likely due to cell proliferation that lowers the amount of modRNA and expressed proteins per cell.

### Example 4 In Vivo Overexpression of PCCA mRNA

FLAG-tagged PCCA modRNA constructs were used to transfect wild-type mice through i.v. injections. 24 hours after the injection, the whole liver lysate and liver mitochondria lysate were prepared as described previously. FLAG-PCCA and total PCCA level was detected by western blot. Vinculiun, a membrane cytoskeletal protein, was used as the loading control for whole liver lysate as described previously. Cytosolic proteins (e.g., GAPDH) and mitochondrial proteins (e.g., COX IV) was followed to ensure the enrichment of mitochondria during fractionation.

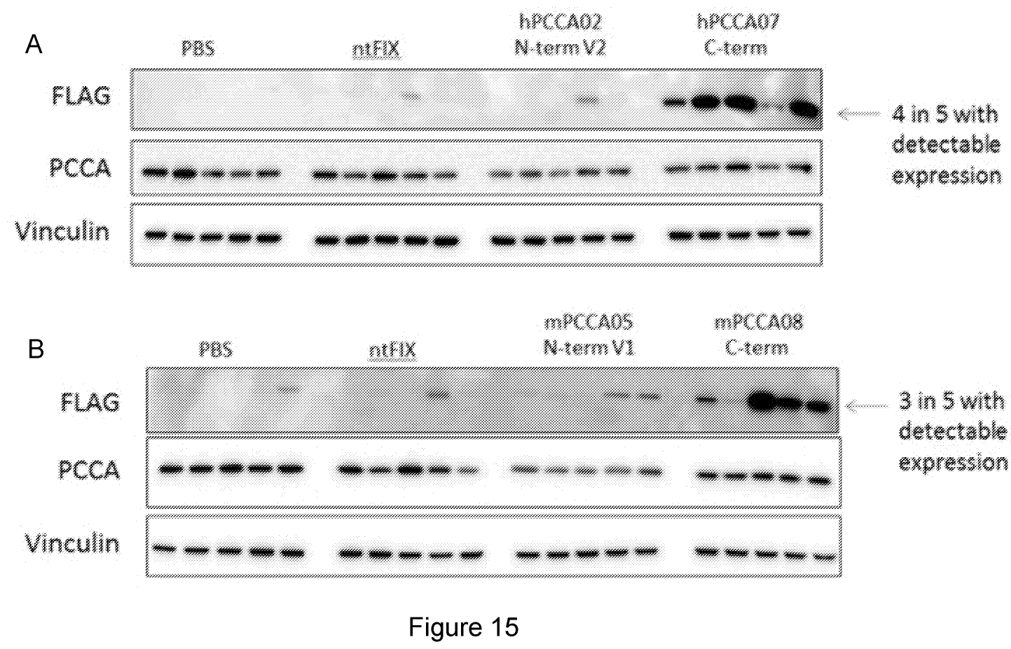

Compared to PBS and ntFIX control groups, most mice dosed with 2.5 mg/kg of MC3-formulated C-FLAG hPCCA or mPCCA showed expression of FLAG-tagged PCCA in the crude liver lysates at 24 hours after injection, detected by anti-FLAG antibody (FIGS. 15A and 15B). However, no obvious increase in total PCCA level was observed in PCCA modRNA-dosed mice, indicating relatively low expression level of exogenous PCCA compared to endogenous PCCA in wild-type mice.

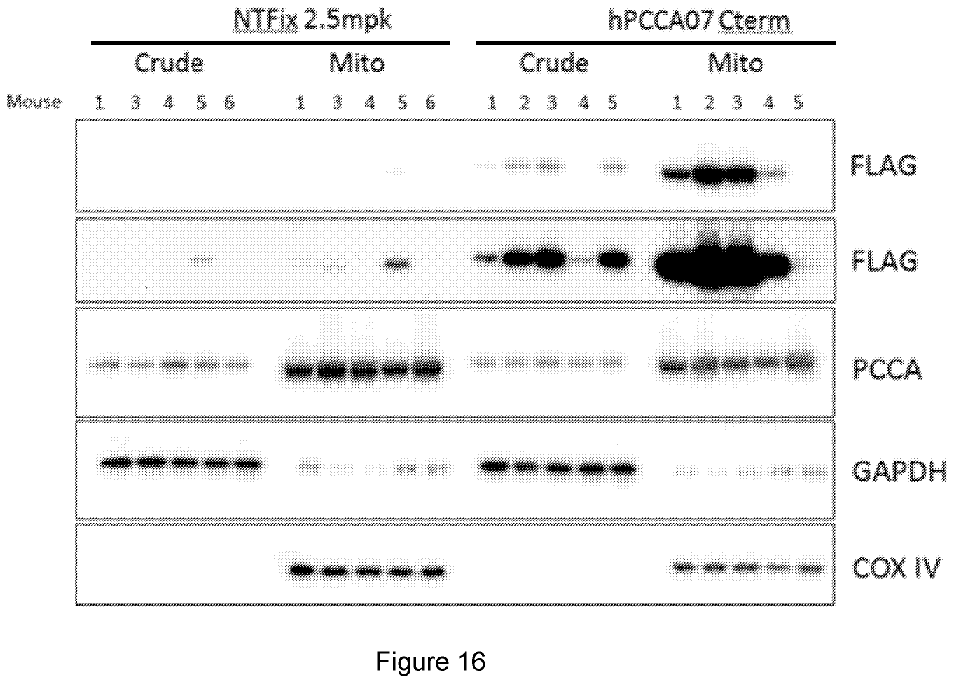

Enrichment of mitochondrial proteins (COX IV) and depletion of cytosolic proteins (GAPDH) was observed after mitochondrial preparation from total liver lysate (FIG. 16). More concentrated signal of C-FLAG PCCA agreed with correct localization to the mitochondria. Clearly, enriched PCCA and Flag-tagged PCCA were detected in liver mitochondrial fractions (FIG. 16).

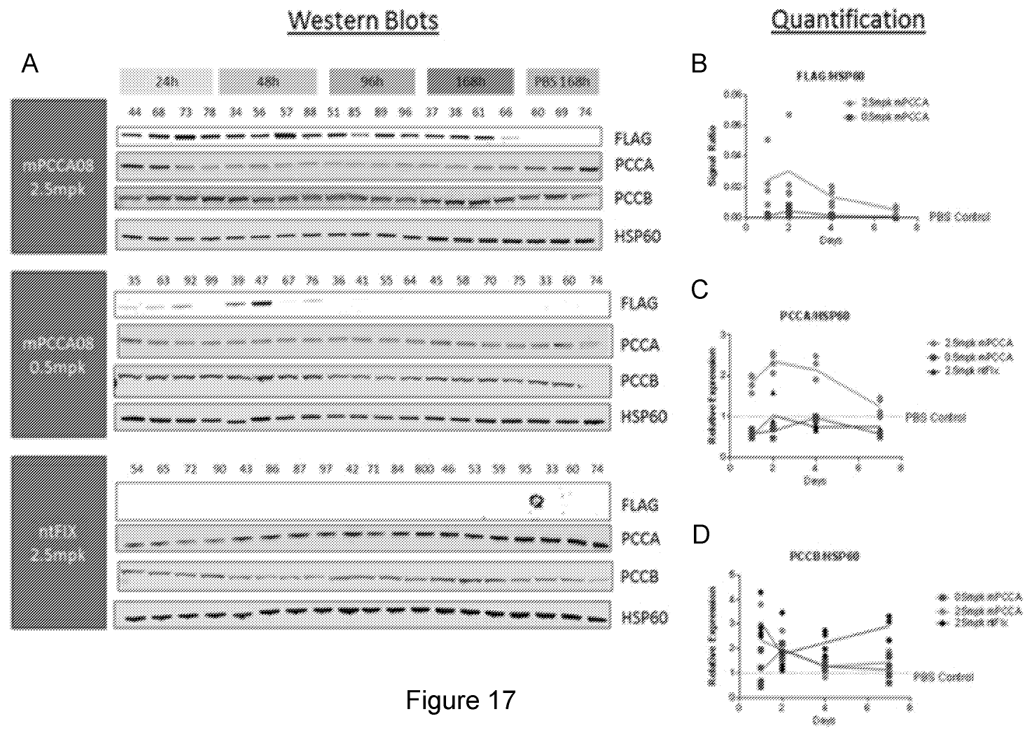

Mouse PCCA modRNAs were also injected through i.v. to wild-type mice. Specifically, liver mitochondria lysate was prepared as described previously. As shown in FIG. 17, C-FLAG PCCA, total PCCA, PCCB level was detected with western blot. Further, mPCCA protein expressed from modRNA was detected in liver mitochondria up to 7 days post i.v. injection. With 2.5 mg/kg dosage, the FLAG signal peaked around Day 2 and slowly went down (FIGS. 17A and 17B). Remarkably, the FLAG signal was still detectable 7 days after injection (FIG. 17A). At 0.5 mg/kg dosage, the FLAG signal was not detectable after 2 days, possibly due to the detection limitation of western blot method (FIGS. 17A and 17B).

Using a mitochondrial heat shock protein (HSP60) as the loading control, quantification of the western blot data suggested injection of wild-type mice at 2.5 mg/kg dosage resulted in a roughly 2-fold increase in total PCCA level (FIG. 17C), but no obvious change in PCCB level above control group (ntFIX) (FIG. 17D).

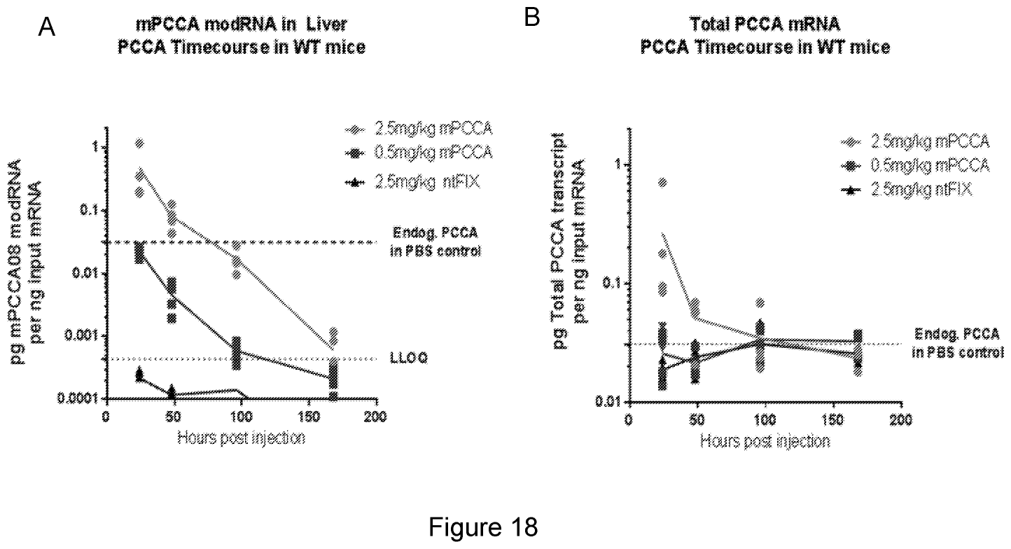

mPCCA modRNA levels were measured by qRT-PCR analysis using modRNA-specific primers and mPCC08 standard curve. As shown in FIG. 18, modRNAs were detectable in a dose-dependent manner. In animals dosed with 2.5 mg/kg mPCCA08-formulated LNPs, mPCCA modRNA was detected at 0.5 pg/ng of total liver mRNA at 24 hours after injection and decreased about 1000 fold at Day 7 after injection (FIG. 18A). Correspondingly, the total PCCA mRNA levels dropped quickly and returned to baseline levels by 96 hours after LNP administration (FIG. 18B).

Despite the ˜1000 decrease in modRNA levels, C-FLAG PCCA protein was still detectable 7 days post injection (FIG. 17), suggesting the long half-life of the expressed PCCA proteins in mouse liver.

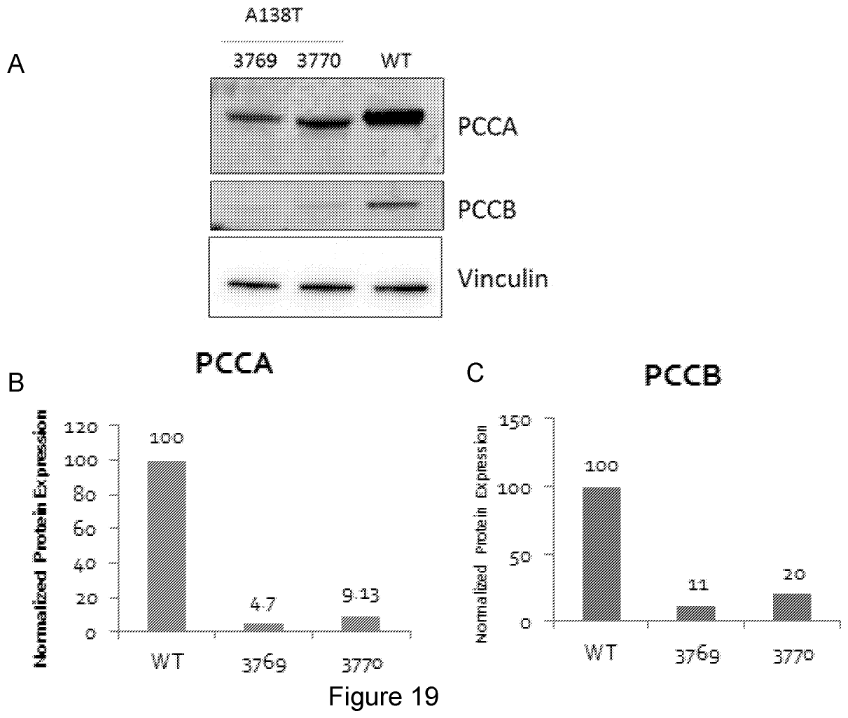

PCCA expression was also tested in an A138T mouse hypomorphic model. As shown in FIG. 19, endogenous PCCA and PCCB expression were dramatically decreased in the A138T hypomorphic mouse.

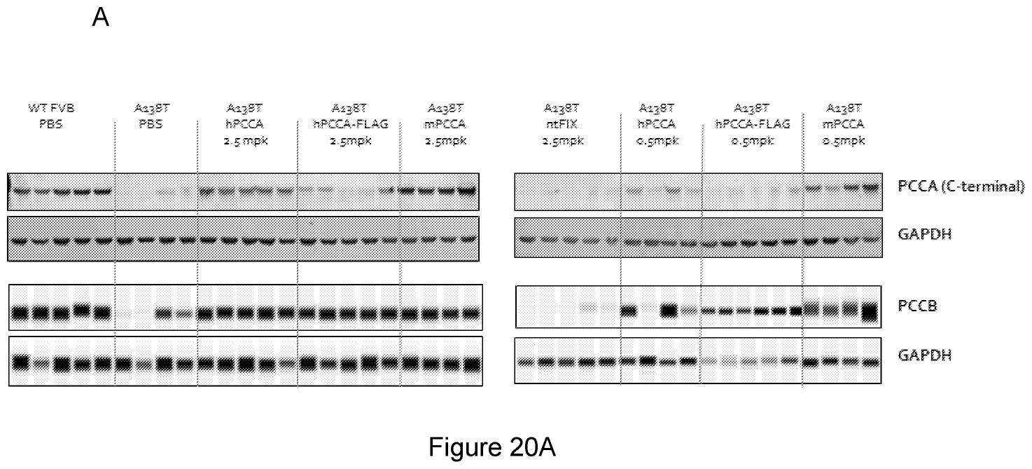

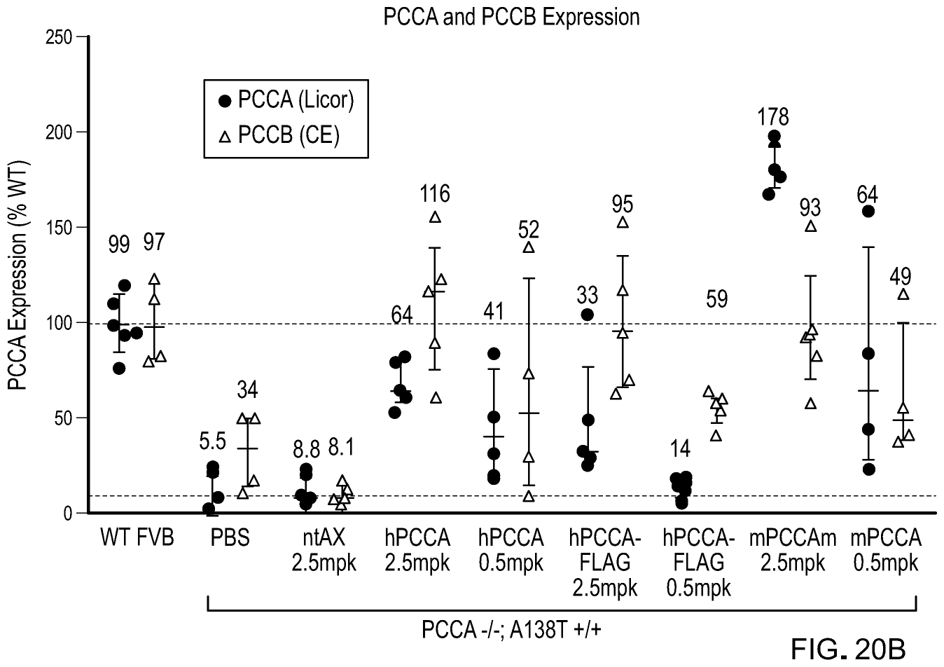

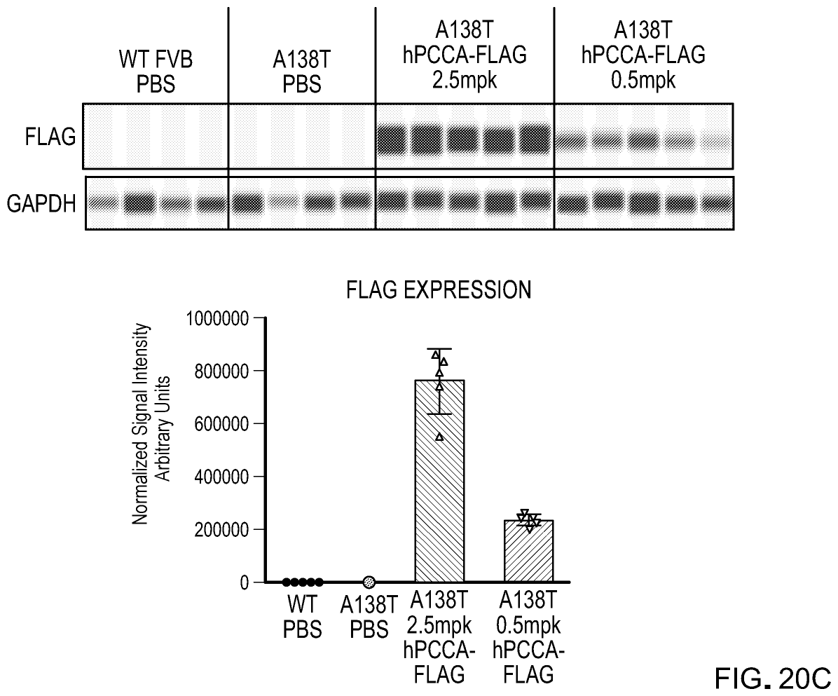

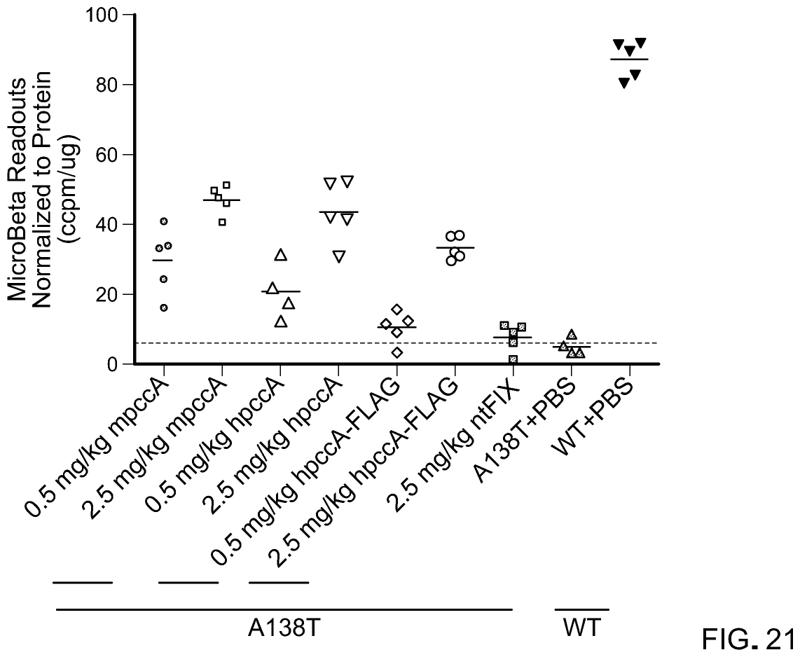

Human and mouse PCCA modRNA constructs (PCCA-LNPs) were injected through i.v. into A138T mice. As shown in FIGS. 20A-20C and FIG. 21, PCCA-LNP constructs increased expression of exogenous untagged and FLAG-tagged human or mouse PCCA proteins in a dosage-dependent manner. In addition, such PCCA modRNA constructs increased endogenous PCCB protein levels, probably due to stabilization of PCCB.

### Example 5 Biomarker Analysis for In Vivo Overexpression of PCCA mRNA

Biomarkers for detecting PCCA in vivo expression and function have been studied. The levels of 2-methylcitric acid (2-MC) and propionylcarnitine (C3) were reported to increase in Propionic Acidemia (PA) settings (Turgeon, C. et al., Clin. Chem., 56:1686-95, 2010). In this Example, the levels of 2-MC and C3 were analyzed with or without PCCA modRNA treatment.

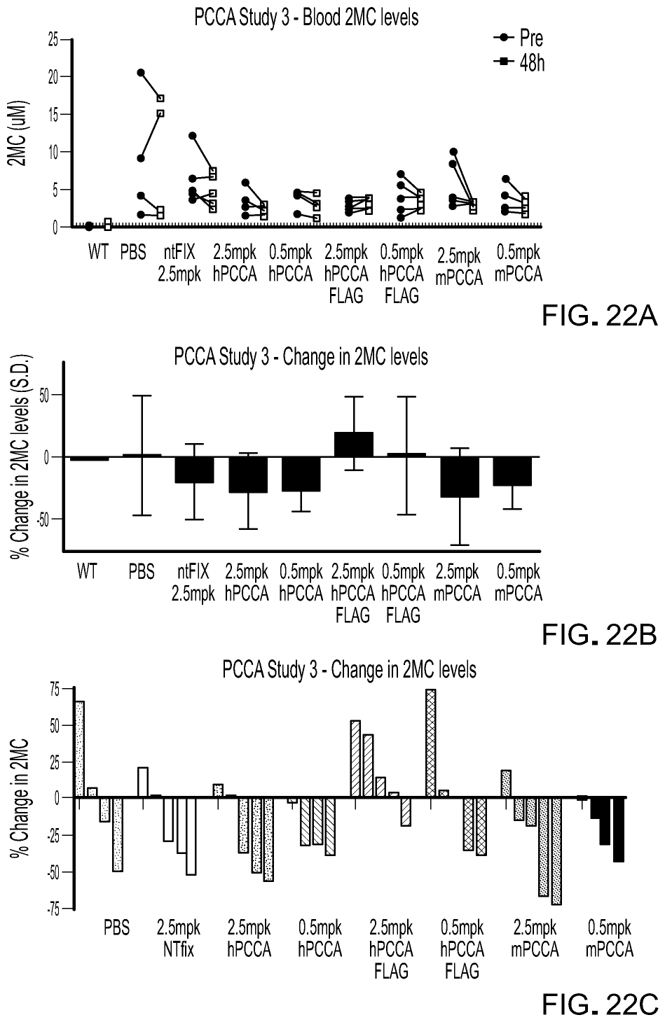

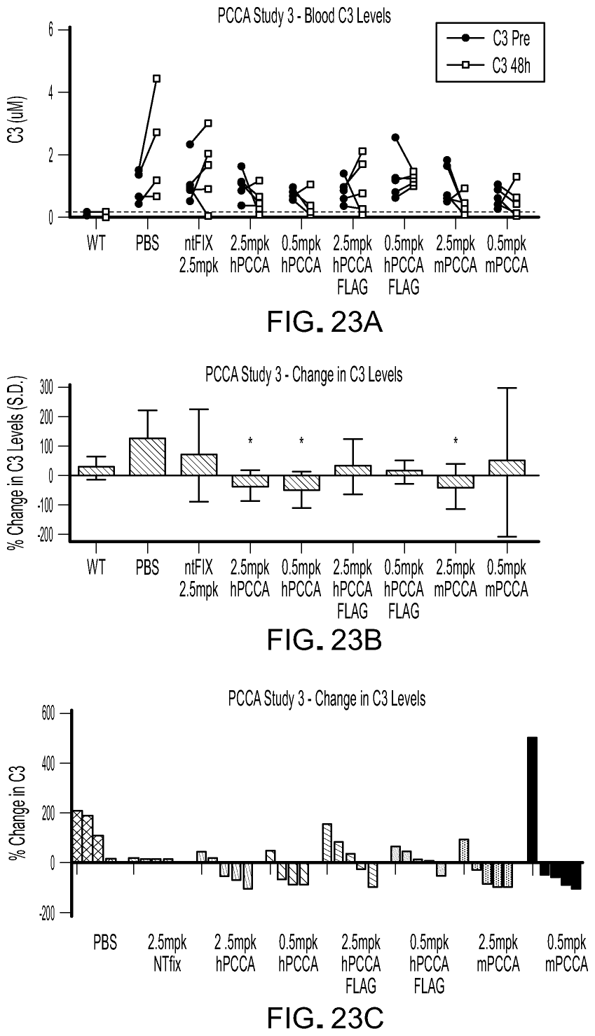

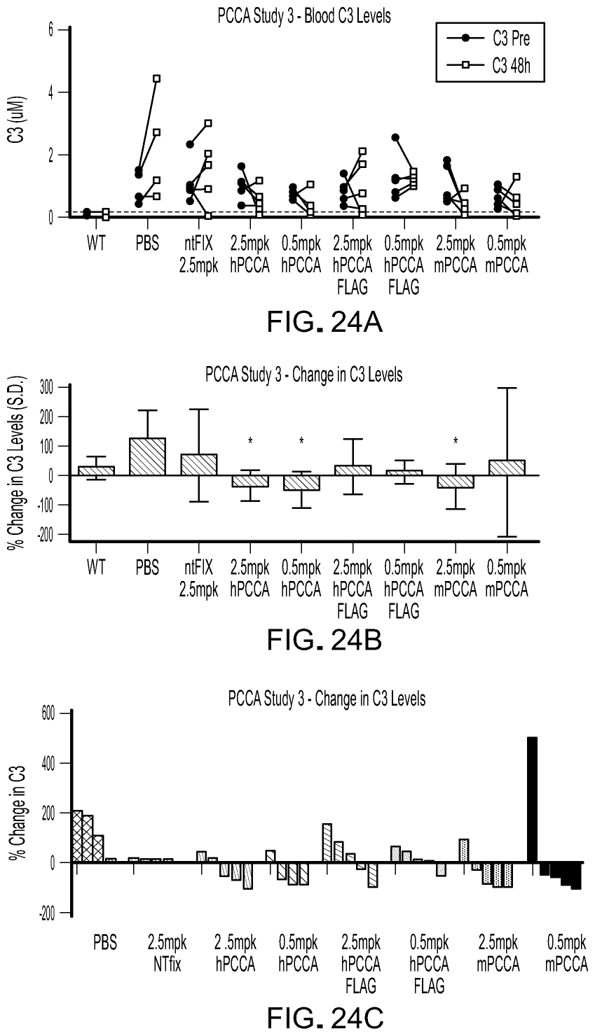

To test the blood levels of these biomarkers, dried blood spot samples were analyzed. As shown in FIGS. 22-24, treatment with different dosages of hPCCA or mPCCA modRNA constructs (untagged or FLAG-tagged) decreased blood levels of 2-MC and C3. Due to high variability of pre-bleed biomarker levels, the comparison of absolute biomarker levels was challenging. Instead, analyses by % change showed a more consistent result-reading. The % Change plots of propionylcarnitine (C3) or propionylcarnitine (C3)/acetylcarnitine (C2) levels in FIGS. 23-24 suggest PD modulation for hPCCA and mPCCA-treated animals. No real evidence of dose-dependent changes was discovered. In addition, the FLAG-tagged hPCCA was less effective to decrease 2-MC and C3 levels than untagged version.

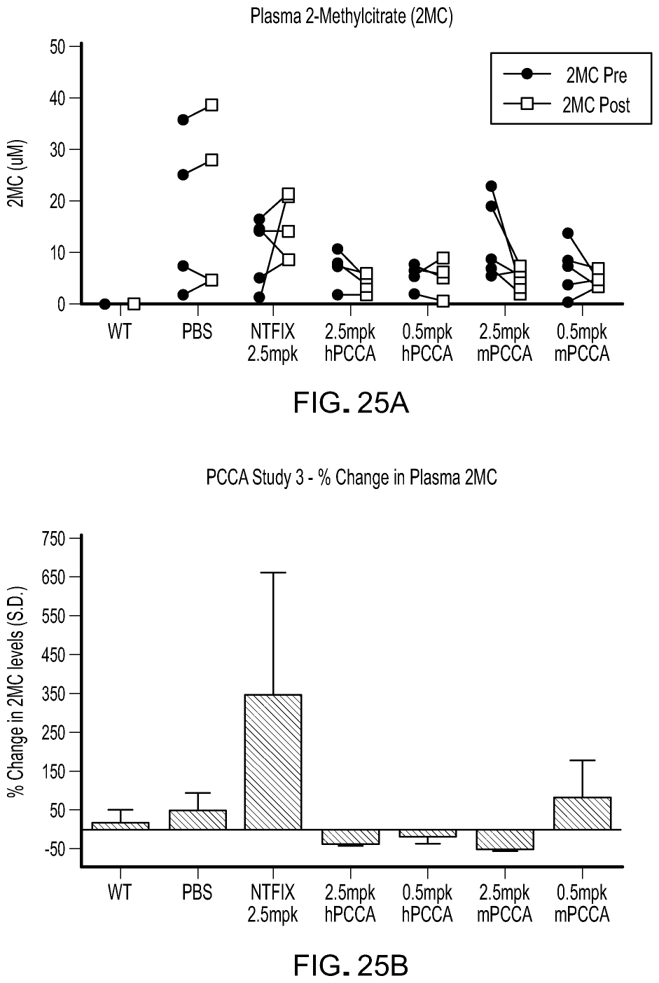

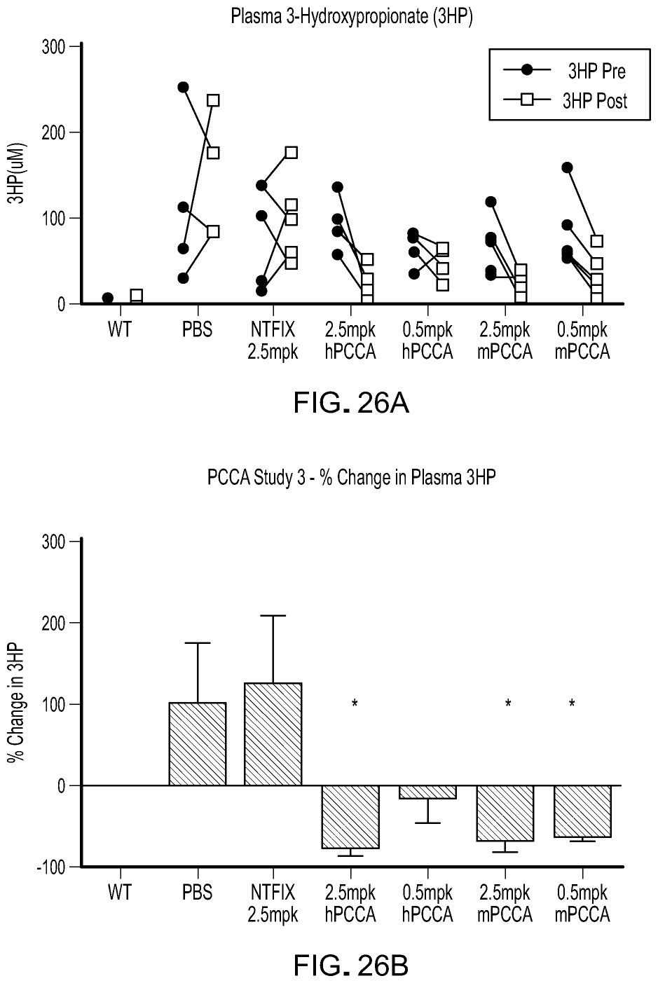

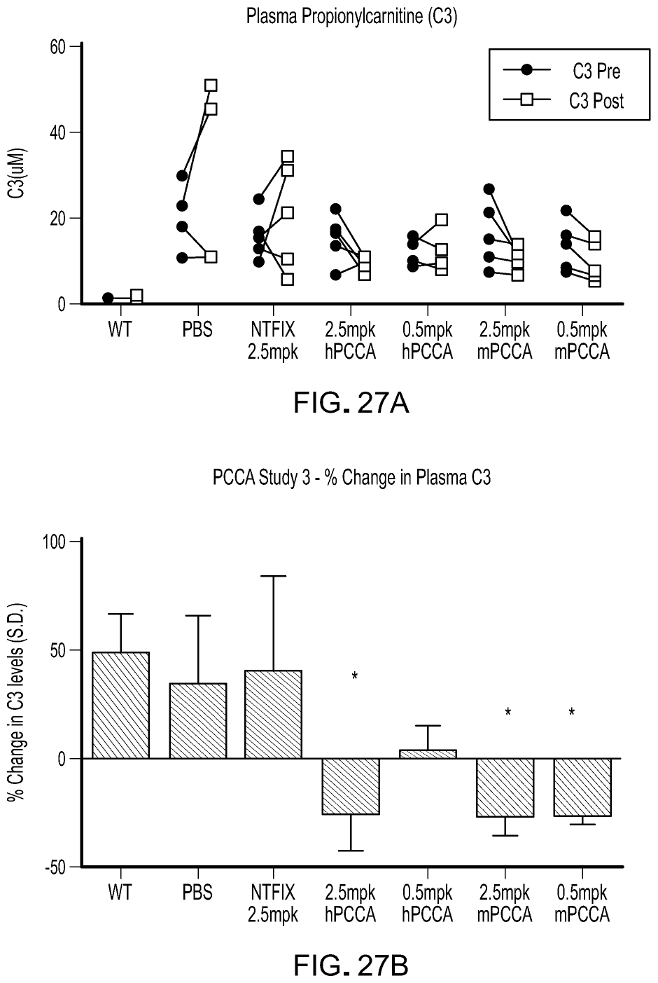

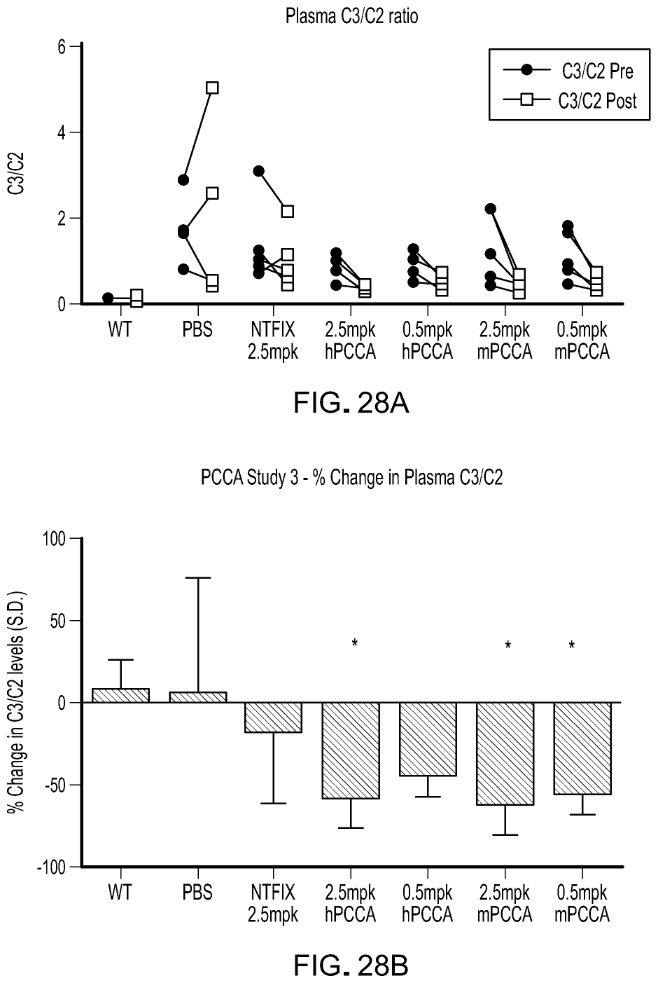

Similarly, the expression levels of plasma biomarkers (such as 2-MC, 3-Hydroxypropionate (3-HP), C3, and C3/C2) were found to be reduced by hPCCA and mPCCA overexpression (FIGS. 25-28).

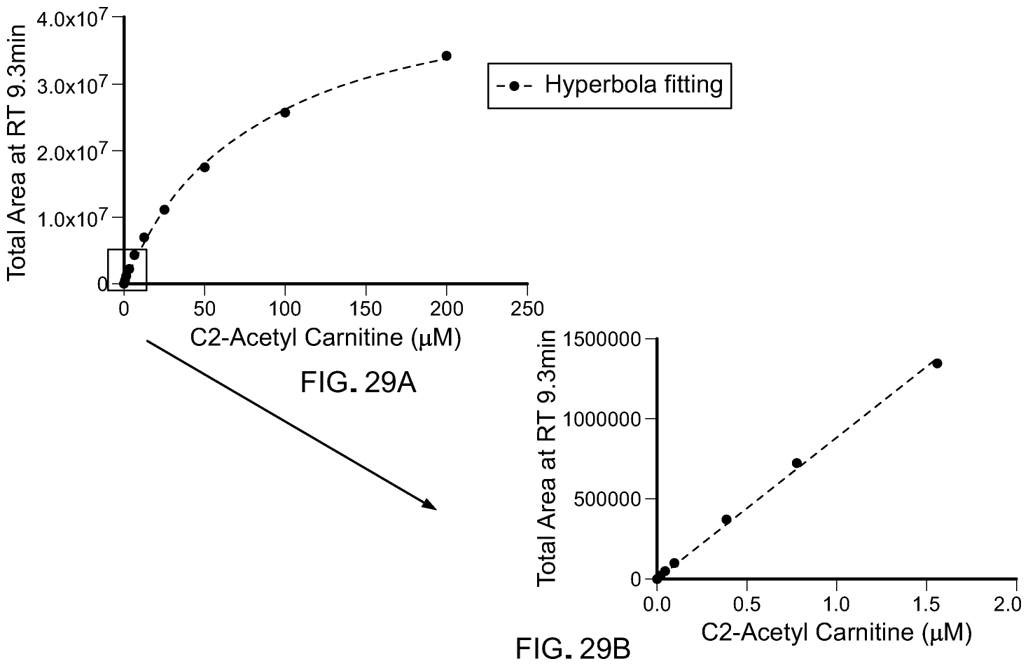

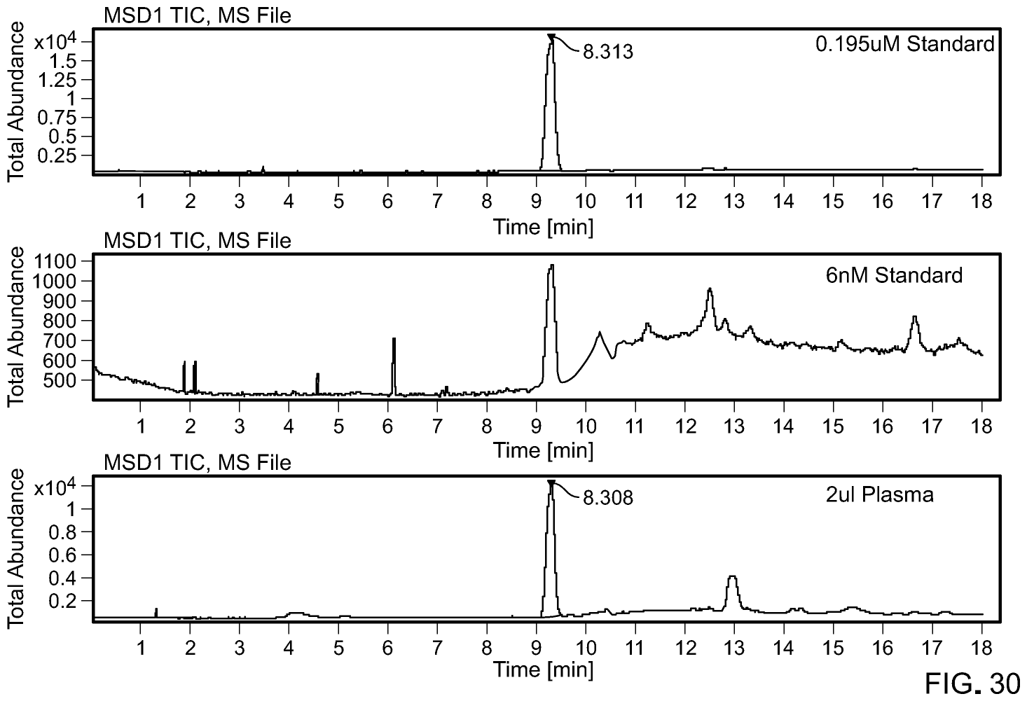

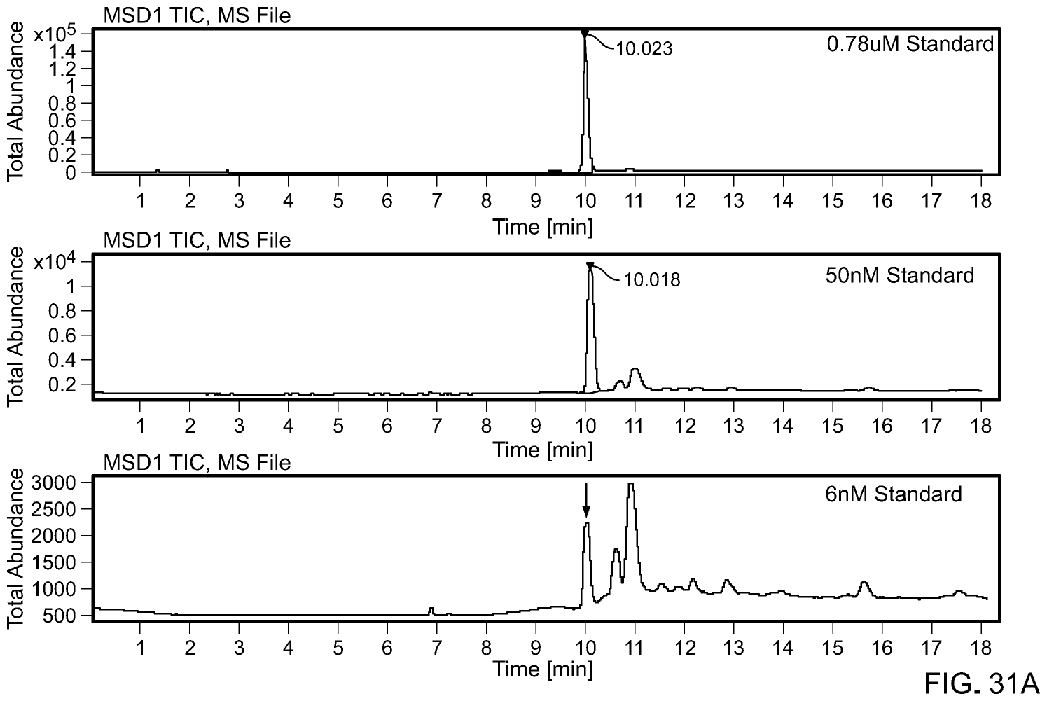

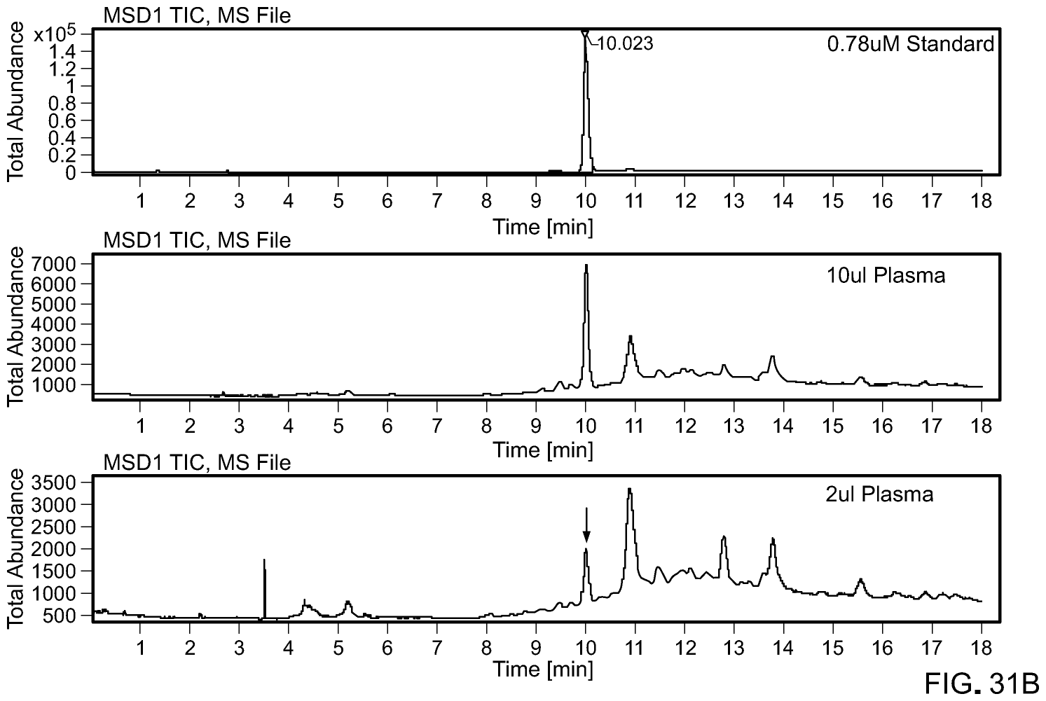

The standard curve of C2 (acetylcarnitine) is shown in FIG. 29. The limit of detection here was about 6 nM (˜1.2 ng/mL). The detection of C2 and C3 by liquid chromatography-mass spectrometry (LC-MS) (SIM) is shown in FIGS. 30-31, respectively.

In conclusion, in this in vivo expression experiment, PCCA modRNA treatment resulted in expression of PCCA protein and probably stabilized PCCB protein. The PCC activity was increased, leading to reduction of circulating 3-HP, C3 and C3/C3 levels. In general, the activity of mPCCA constructs was better than hPCCA constructs, while the hPPCA-FLAG constructs had the least expression/activity.

### Example 6 Dosage Effect of PCCA mRNA Treatment

A138T hypomorphic mice were treated with lipid nanoparticle (LNP) encapsulated modRNA encoding human pccA through single dose administration. Biomarker levels will be measured to analyze the dose response.

Specifically, mixed gender and age-matched mice of about 12-16 weeks of age were used for a single-dose study with hPCCA modRNA treatment. Groups of mice were treated with 0.5 mg/kg GFP-LNP, 0.5 mg/kg hPCCA LNP-modRNA, 0.25 mg/kg hPCCA LNP-modRNA, or 0.125 mg/kg hPCCA LNP-modRNA. Mice were pre-bled five days prior to the treatment day. On the treatment day these different constructs were administered through IV injection. DBS were performed at 1, 3, 7, 11, 14, 18 and 21 days after administration.

Similarly, mPCCA modRNA constructs encapsulated in LNP are prepared and administered to A138T hypomorphic mice in a multiple-dose study. After the first dosage at Day 0, further dosages are given at Day 7, 14, 21 or 28.

For read-outs, levels of propionylcarnitine (C3/C2) and methylcitrate (MetCit) from dry blood spot (MPI) assay are used as the primary outcome measurements. Levels of 3-HP from plasma (UCSD), PCCA expression on whole liver, and PCCA activity on whole liver homogenates are secondary outcome measurements.

### Example 7 Co-Expression of PCCA and PCCB mRNA Constructs

A138T hypomorphic mice were treated with lipid nanoparticle (LNP) encapsulated modRNA encoding PCCA only or together with modRNA encoding PCCA PCCB. Specifically, female mice of mixed ages were dosed with 0.5 mg/kg hPCCA modRNA, 0.5 mg/kg hPCCB modRNA, 0.5 mg/kg hPCCA modRNA+0.5 mg/kg hPCCB modRNA, 0.3 mg/kg hPCCA modRNA, 0.3 mg/kg hPCCA modRNA+0.3 mg/kg hPCCB modRNA, 0.15 mg/kg hPCCA modRNA, 0.15 mg/kg hPCCA modRNA+0.15 mg/kg hPCCA modRNA, or PBS control. Forty-eight hours after dosing blood were collected and liver tissues were harvested from the mice.

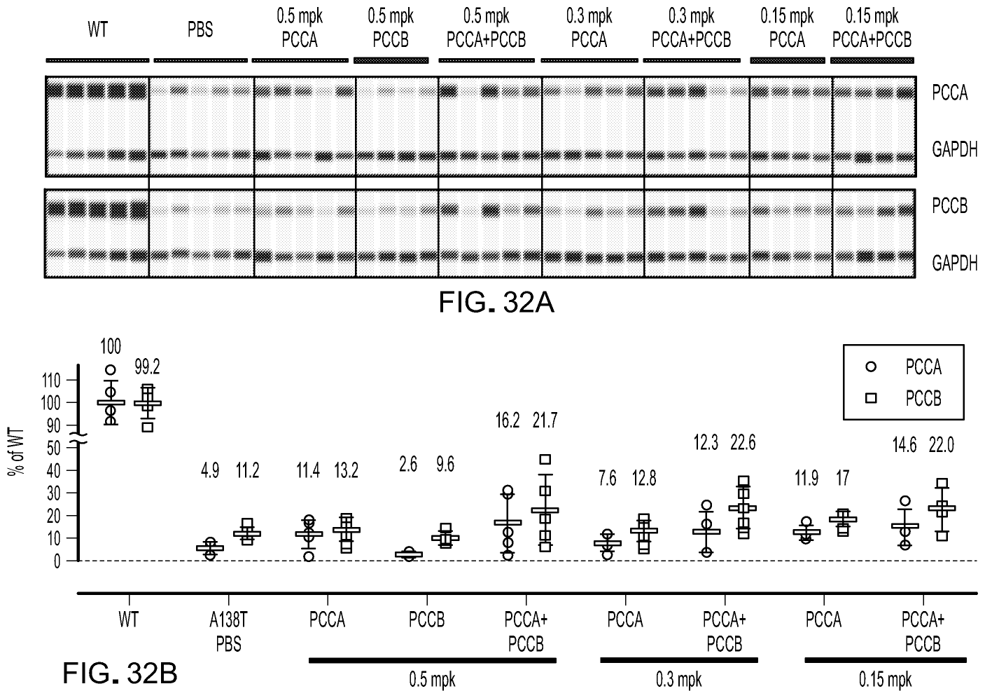

Liver protein levels for PCCA and PCCB were analyzed as described herein. As shown in FIG. 32, hPCCA and hPCCB modRNA constructs improved their protein levels, respectively. Administering only hPCCB modRNA increased hPCCB protein levels moderately. However, co-administration of hPCCA and hPCCB modRNA constructs further increased hPCCB protein levels, probably due to the stabilization effect of PCCA as already seen in in vitro and in vivo studies.

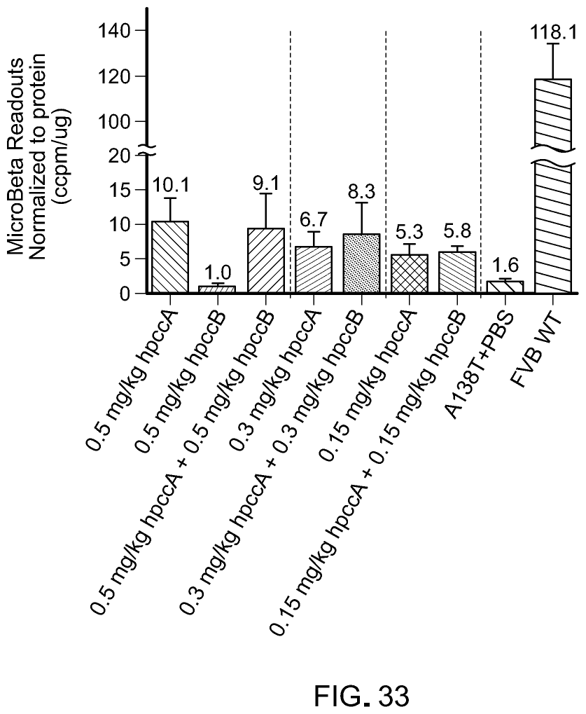

Treatment with 0.5 mg/kg hPCCA modRNA improved PCC activity about 6.3 fold from the baseline level (from a readout of about 1.6 for PBS control to about 10.1 for PCCA in FIG. 33). Such PCC activity was about 8.6% of the wild-type activity (i.e., a readout of about 10.1 for PCCA versus about 118.1 for wild-type).

### Other Embodiments

It is understood that while the invention has been described in conjunction with the detailed description thereof, the foregoing description is intended to illustrate and not limit the scope of the invention, which is defined by the scope of the appended claims. The materials, methods, and examples are illustrative only and not intended to be limiting. All publications, patent applications, patents, sequences, database entries and other references cited and described herein are incorporated by reference in their entireties. Other aspects, advantages and modifications are within the scope of the following claims.

What is claimed is:

1. A method of treating propionic acidemia in a patient in need thereof comprising administering to the patient a therapeutically effective amount of a composition comprising a modified mRNA molecule encoding a propionyl-CoA carboxylase polypeptide.

2. The method of claim 1 wherein the modified mRNA molecule encoding a polypeptide comprises at least one of a propionyl-CoA carboxylase alpha chain protein or a propionyl-CoA carboxylase beta chain protein.

3. The method of claim 1 wherein the modified mRNA molecule comprises at least one modified nucleoside.

4. The method of claim 3, wherein the at least one modified nucleoside is selected from the group consisting of: pseudouridine, 1-methyl-pseudouridine, 5-methylcytidine, 5-methyluridine, 2′-O-methyluridine, 2-thiouridine, 5-methoxyuridine and N6-methyladenosine.

5. The method of claim 1, wherein the modified mRNA molecule comprises a poly(A) tail, a Kozak sequence, a 3′ untranslated region, a 5′ untranslated region or any combination thereof.

6. The method of claim 1, wherein the modified mRNA molecule encodes a PCCA subunit comprising a sequence selected from the group consisting of SEQ ID NOS:1-3.

7. The method of claim 1, wherein the modified mRNA molecule encodes a PCCB subunit comprising a sequence of SEQ ID NO:4 or SEQ ID NO:5.

8. The method of claim 1, wherein the modified mRNA is encapsulated in a lipid nanoparticle.

9. A pharmaceutical composition comprising a therapeutically effective amount of a modified mRNA molecule wherein the modified mRNA molecule encodes one or both of a propionyl-CoA carboxylase subunit.

10. The pharmaceutical composition of claim 9, wherein the proprionyl-CoA carboxylase is an alpha chain protein comprising the amino acid sequence selected from the group consisting of SEQ ID NOS:1-3, and a pharmaceutically acceptable carrier, diluent or excipient.

11. The pharmaceutical composition of claim 9, wherein the proprionyl-CoA carboxylase is an beta chain protein comprising the amino acid sequence of SEQ ID NO:4 or SEQ ID NO:5, and a pharmaceutically acceptable carrier, diluent or excipient.
